SEQUENCE LISTING (36 sequences)

SEQ ID NO: 1 - protein, 9 aa, Artificial
  Env-epitope
ERYLKDQQL

SEQ ID NO: 2 - protein, 9 aa, Artificial
  HIV Rev67-75 epitope
SAEPVPLQL

SEQ ID NO: 3 - protein, 9 aa, Artificial
  NP418-426 epitope
LPFEKSTVM

SEQ ID NO: 4 - protein, 9 aa, Artificial
  NP44-52 epitope
CTELKLSDY

SEQ ID NO: 5 - protein, 9 aa, Artificial
  M58-66 epitope
GILGFVFTL

SEQ ID NO: 6 - DNA, 44 bp, Human immunodeficiency virus
  5′ part of multiple cloning site
gctagcgtttaaacttaagcttggtaccgagctcggatcctatg

SEQ ID NO: 7 - DNA, 22 bp, Human immunodeficiency virus
  3′ part of multiple cloning site
agatccaccggtcgccaccatg

SEQ ID NO: 8 - DNA, 91 bp, Human immunodeficiency virus
  multiple cloning site
gctagcgctaccggactcagatctcgagctcaagcttcgaattctgcagtcgacggtaccgcgggcccgggatccaccggtcgccaccatg

SEQ ID NO: 9 - DNA, 54 bp, Influenza virus
  spacer
gatctcgaggccaccatggagagatacctaaaggatcaagatatccgggatcca

SEQ ID NO: 10 - DNA, 15 bp, Influenza virus
  5′ part of multiple cloning site
ctcgagacgcgtatg

SEQ ID NO: 11 - DNA, 30 bp, Influenza virus
  3′ part of multiple cloning site
gttatccgggatccaccggtcgccaccatg

SEQ ID NO: 12 - protein, 9 aa, Artificial
  Derived from influenza virus genes encoding NP strain A/NL/18/94 (NP01), NP strain A/HK/2/68 (NP02), NP strain A/ PR/8/34 (NP03)
CTELKLSDY

SEQ ID NO: 13 - protein, 9 aa, Artificial
  Derived from influenza virus genes encoding NP strain A/NL/18/94 (NP01)
LPFEKSTVM

SEQ ID NO: 14 - protein, 9 aa, Artificial
  Derived from influenza virus genes encoding NP strain A/HK/2/68 (NP02)
LPFDKPTIM

SEQ ID NO: 15 - protein, 9 aa, Artificial
  Derived from influenza virus genes encoding NP strain A/PR/8/34 (NP03)
LPFDRTTIM

SEQ ID NO: 16 - protein, 9 aa, Artificial
  Derived from influenza virus genes encoding M1 strain A/NL/18/94 (M1)
GILGFVFTL

SEQ ID NO: 17 - DNA, 1509 bp, Human immunodeficiency virus type 1
  CDS (1)..(1509): Codon optimized for Homo sapiens
atgggcgccagagccagcgtgctgtctggcggcgagctggacagatgggagaagatcaggctgagacctggcggcaagaagaagtacaagctgaagcacattgtgtgggccagcagagagctggagagattcgccgtgaaccctggcctgctggagaccagcgagggctgtagacagatcctgggccagctgcagcctagcctgcagaccggcagcgaggagctgagaagcctgtacaacaccgtggccaccctgtactgtgtgcaccagaagatcgaggtgaaggacaccaaggaggccctggacaagatcgaggaggagcagaacaagtccaagaagaaggcccagcaggccgctgccgacaccggcaacagcagccaggtgtcccagaactaccccatcgtgcagaacctgcagggccagatggtgcaccaggccatcagccctagaaccctgaacgcctgggtgaaggtggtggaggagaaggccttcagccctgaggtgatccctatgttcagcgccctgagcgagggcgccacccctcaggacctgaacaccatgctgaacacagtgggcggccaccaggccgccatgcagatgctgaaggagaccatcaacgaggaggccgccgagtgggacagactgcaccctgtgcacgctggccctatcgcccctggccagatgagagagcctaggggcagcgacatcgccggcaccacaagcaccctgcaggaacagatcggctggatgaccaacaacccccctatccctgtgggcgaaatctacaagcggtggatcatcctgggcctgaacaagattgtgcggatgtacagccctaccagcatcctggacatcagacagggccccaaggagcccttcagagactacgtggaccggttctacaagaccctgagagccgagcaggccagccaggaagtgaagaactggatgaccgagaccctgctggtgcagaacgccaaccctgactgcaagaccatcctgaaggccctgggccctgccgccaccctggaggagatgatgaccgcctgccagggagtgggcggacctggccacaaggccagagtgctggccgaggccatgagccaggtgaccaacagcgccaccatcatgatgcagaggggcaacttcaggaaccagcgcaagaccgtgaagtgcttcaactgcggcaaggagggccacatcgccaagaactgcagagcccccagaaagaagggctgctggaagtgtggaaaagagggacaccagatgaaggactgcaccgagaggcaggccaacttcctgggcaagatttggcctagccacaagggcagacccggcaacttcctgcagagcaggcctgagcctaccgcccctcctgaggagagcttcagattcggcgaggagaccaccacccctagccagaagcaggagcccatcgacaaggagctgtaccctctggccagcctgagatccctgttcggcaacgaccctagcagccaagatctttag

SEQ ID NO: 18 - protein, 502 aa, Human immunodeficiency virus type 1
MGARASVLSGGELDRWEKIRLRPGGKKKYKLKHIVWASRELERFAVNPGLLETSEGCRQILGQLQPSLQTGSEELRSLYNTVATLYCVHQKIEVKDTKEALDKIEEEQNKSKKKAQQAAADTGNSSQVSQNYPIVQNLQGQMVHQAISPRTLNAWVKVVEEKAFSPEVIPMFSALSEGATPQDLNTMLNTVGGHQAAMQMLKETINEEAAEWDRLHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQVTNSATIMMQRGNFRNQRKTVKCFNCGKEGHIAKNCRAPRKKGCWKCGKEGHQMKDCTERQANFLGKIWPSHKGRPGNFLQSRPEPTAPPEESFRFGEETTTPSQKQEPIDKELYPLASLRSLFGNDPSSQDL

SEQ ID NO: 19 - DNA, 621 bp, Human immunodeficiency virus type 1
  CDS (1)..(621): Codon optimized for Homo sapiens
atgggcggcaagtggagcaagagaagcgtggtgggctggcctacagtgagggagaggatgagaagagccgagcctgccgccgacggagtgggcgccgtgtccagagacctggagaagcacggcgccatcaccagcagcaacaccgccgccaacaacgccgactgcgcctggctggaggcccaggaggaggaggaagtgggcttccctgtgagacctcaggtgcccctgagacccatgacctacaaggccgccgtggacctgagccacttcctgaaggagaagggcggcctggagggcctgatctacagccagaagcggcaggacatcctggacctgtgggtgtaccacacccagggctacttccctgactggcagaactacacccctggccctggcatcagataccctctgaccttcggctggtgcttcaagctggtgcctgtggagcctgagaaggtggaggaggccaacgagggcgagaacaactctctgctgcaccctatgagcctgcacggcatggacgaccctgagagagaggtgctggtgtggaagttcgacagcaggctggccttccaccacatggccagagagctgcaccccgagtactacaaagatctgtga

SEQ ID NO: 20 - protein, 206 aa, Human immunodeficiency virus type 1
MGGKWSKRSVVGWPTVRERMRRAEPAADGVGAVSRDLEKHGAITSSNTAANNADCAWLEAQEEEEVGFPVRPQVPLRPMTYKAAVDLSHFLKEKGGLEGLIYSQKRQDILDLWVYHTQGYFPDWQNYTPGPGIRYPLTFGWCFKLVPVEPEKVEEANEGENNSLLHPMSLHGMDDPEREVLVWKFDSRLAFHHMARELHPEYYKDL

SEQ ID NO: 21 - DNA, 621 bp, Human immunodeficiency virus type 1
  CDS (1)..(621): Codon optimized for Homo sapiens
atggccggcaagtggagcaagagaagcgtggtgggctggcctacagtgagggagaggatgagaagagccgagcctgccgccgacggagtgggcgccgtgtccagagacctggagaagcacggcgccatcaccagcagcaacaccgccgccaacaacgccgactgcgcctggctggaggcccaggaggaggaggaagtgggcttccctgtgagacctcaggtgcccctgagacccatgacctacaaggccgccgtggacctgagccacttcctgaaggagaagggcggcctggagggcctgatctacagccagaagcggcaggacatcctggacctgtgggtgtaccacacccagggctacttccctgactggcagaactacacccctggccctggcatcagataccctctgaccttcggctggtgcttcaagctggtgcctgtggagcctgagaaggtggaggaggccaacgagggcgagaacaactctgccgcccaccctatgagcctgcacggcatggacgaccctgagagagaggtgctggtgtggaagttcgacagcaggctggccttccaccacatggccagagagctgcaccccgagtactacaaagatctgtga

SEQ ID NO: 22 - protein, 206 aa, Human immunodeficiency virus type 1
MAGKWSKRSVVGWPTVRERMRRAEPAADGVGAVSRDLEKHGAITSSNTAANNADCAWLEAQEEEEVGFPVRPQVPLRPMTYKAAVDLSHFLKEKGGLEGLIYSQKRQDILDLWVYHTQGYFPDWQNYTPGPGIRYPLTFGWCFKLVPVEPEKVEEANEGENNSAAHPMSLHGMDDPEREVLVWKFDSRLAFHHMARELHPEYYKDL

SEQ ID NO: 23 - DNA, 1494 bp, Influenza A virus
  CDS (1)..(1494): Nucleoprotein from Influenza strain NL-18-94
atggcgtcccaaggcaccaaacggtcttatgaacagatggaaactgatggggaacgccagaatgcaactgagattagggcatccgtcgggaagatgattgatggaattgggcgattctacatccaaatgtgcactgaacttaaactcagtgattatgaagggcggttgatccagaacagcttgacaatagagaaaatggtgctctctgcttttgatgagagaaggaatagatatctggaagaacaccccagcgcggggaaagatcctaagaaaactggagggcccatatacaagagagtagatggaagatggatgagggaactcgtcctttatgacaaagaagaaataaggcgaatctggcgccaagccaacaatggtgaggatgcgacagctggtctaactcacatgatgatctggcattccaatttgaatgatacaacataccagaggacaagagctcttgttcgcaccggaatggatcccaggatgtgctctctgatgcagggttcgactctccctagaaggtccggagctgcaggtgctgcagtcaaaggaatcgggacaatggtgatggagctgatcagaatggtcaaacgggggatcaacgatcgaaatttctggagaggtgagaatgggcggaaaacaaggagtgcttatgaaagaatgtgcaacattcttaaaggaaaatttcaaacagctgcacaaagagcaatgatggatcaagtgagagaaagccggaacccaggaaatgctgagatcgaagatctcatatttttggcaagatctgcattaatattgagagggtcagttgctcacaaatcttgcctacctgcctgtgtgtatggacctgcagtatccagtgggtacgacttcgaaaaagagggatattccttagtgggaatagaccctttcaaactacttcaaaatagccaagtatacagcctaatcagaccaaacgagaatccagcacacaagagtcagctggtgtggatggcatgccattctgctgcatttgaagatttgagattgttaagcttcatcagagggaccaaagtatctccgcgggggaaactttcaactagaggagtacaaattgcttcaaatgagaacatggataatatgggatcaagtactcttgaactgagaagcgggtactgggccataaggaccaggagtggaggaaacactaatcaacagagggcctccgcaggccaaatcagtgtgcaacctacgttttctgtacaaagaaacctcccatttgaaaagtcaaccgtcatggcagcattcactggaaatacggagggaagaacctcagacatgagggcagaaatcataagaatgatggaaggtgcaaaaccagaagaagtgtccttccgtgggcggggagttttcgagctctcagacgagaaggcaacgaacccgatcgtgccctcttttgacatgagtaatgaaggatcttatttcttcggagacaatgcagaggagtacgacaat

SEQ ID NO: 24 - protein, 498 aa, Influenza A virus
MASQGTKRSYEQMETDGERQNATEIRASVGKMIDGIGRFYIQMCTELKLSDYEGRLIQNSLTIEKMVLSAFDERRNRYLEEHPSAGKDPKKTGGPIYKRVDGRWMRELVLYDKEEIRRIWRQANNGEDATAGLTHMMIWHSNLNDTTYQRTRALVRTGMDPRMCSLMQGSTLPRRSGAAGAAVKGIGTMVMELIRMVKRGINDRNFWRGENGRKTRSAYERMCNILKGKFQTAAQRAMMDQVRESRNPGNAEIEDLIFLARSALILRGSVAHKSCLPACVYGPAVSSGYDFEKEGYSLVGIDPFKLLQNSQVYSLIRPNENPAHKSQLVWMACHSAAFEDLRLLSFIRGTKVSPRGKLSTRGVQIASNENMDNMGSSTLELRSGYWAIRTRSGGNTNQQRASAGQISVQPTFSVQRNLPFEKSTVMAAFTGNTEGRTSDMRAEIIRMMEGAKPEEVSFRGRGVFELSDEKATNPIVPSFDMSNEGSYFFGDNAEEYDN

SEQ ID NO: 25 - DNA, 1494 bp, Influenza A virus
  CDS (1)..(1494): Nucleoprotein from Influenza strain HK/2/68 (H3N2)
atggcgtcccaaggcaccaaacggtcttatgaacagatggaaactgatggggaacgccagaatgcaactgagatcagagcatccgtcgggaagatgattaatggaattggacgattctacatccaaatgtgcactgaacttaaactcagtgattatgaggggcgactgatccagaacagcttaacaatagagagaatggtgctctctgcttttgacgaaagaaggaataaatatctggaagaacatcccagcgcggggaaggatcctaagaaaactggaggacccatatacaagagagtagatggaaagtggatgagggaactcgtcctttatgacaaagaagaaataaggcgaatctggcgccaagccaataatggtgatgatgcaacagctggtctgactcacatgatgatctggcattccaatttgaatgatacaacataccagaggacaagagctcttgttcgcaccggcatggatcccaggatgtgctctctgatgcagggttcgactctccctaggaggtctggagctgcaggcgctgcagtcaaaggagttgggacaatggtgatggagttgataaggatgatcaaacgtgggatcaatgatcggaacttctggagaggtgaaaatggacgaaaaacaaggagtgcttacgagagaatgtgcaacattctcaaaggaaaatttcaaacagctgcacaaagggcaatgatggatcaagtgagagaaagtcggaacccaggaaatgctgagatcgaagatctcatctttctggcacggtctgcactcatattgagagggtcagttgctcacaaatcttgtctgcccgcctgtgtgtatggacctgccgtagccagtggctacgacttcgaaaaagagggatactctttagtgggaatagaccctttcaaactgcttcaaaacagccaagtatacagcctaatcagaccgaacgagaatccagcacacaagagtcagctggtgtggatggcatgcaattctgctgcatttgaagatctaagagtattaagcttcatcagagggaccaaagtatccccaagggggaaactttccactagaggagtacaaattgcttcaaatgaaaacatggatgctatggaatcaagtactcttgaactgagaagcaggtactgggccataagaaccagaagtgggggaaacactaatcaacagagggcctctgcaggtcaaatcagtgtgcaacctgcattttctgtgcaaagaaacctcccatttgacaaaccaaccatcatggcagcattcactgggaatacagagggaagaacatcagacatgagggcagaaattataaggatgatggaaggtgcaaaaccagaagaaatgtccttccaggggcggggagtcttcgagctctcggacgaaaaggcagcgaacccgatcgtgccctcttttgacatgagtaatgaaggatcttatttcttcggagacaatgcagaggagtacgacaat

SEQ ID NO: 26 - protein, 498 aa, Influenza A virus
MASQGTKRSYEQMETDGERQNATEIRASVGKMINGIGRFYIQMCTELKLSDYEGRLIQNSLTIERMVLSAFDERRNKYLEEHPSAGKDPKKTGGPIYKRVDGKWMRELVLYDKEEIRRIWRQANNGDDATAGLTHMMIWHSNLNDTTYQRTRALVRTGMDPRMCSLMQGSTLPRRSGAAGAAVKGVGTMVMELIRMIKRGINDRNFWRGENGRKTRSAYERMCNILKGKFQTAAQRAMMDQVRESRNPGNAEIEDLIFLARSALILRGSVAHKSCLPACVYGPAVASGYDFEKEGYSLVGIDPFKLLQNSQVYSLIRPNENPAHKSQLVWMACNSAAFEDLRVLSFIRGTKVSPRGKLSTRGVQIASNENMDAMESSTLELRSRYWAIRTRSGGNTNQQRASAGQISVQPAFSVQRNLPFDKPTIMAAFTGNTEGRTSDMRAEIIRMMEGAKPEEMSFQGRGVFELSDEKAANPIVPSFDMSNEGSYFFGDNAEEYDN

SEQ ID NO: 27 - DNA, 1494 bp, Influenza A virus
  CDS (1)..(1494): Nucleoprotein from Influenza strain PR/8/34
atggcgtcccaaggcaccaaacggtcttacgaacagatggagactgatggagaacgccagaatgccactgaaatcagagcatccgtcggaaaaatgattggtggaattggacgattctacatccaaatgtgcaccgaactcaaactcagtgattatgagggacggttgatccaaaacagcttaacaatagagagaatggtgctctctgcttttgacgaaaggagaaataaatacctggaagaacatcccagtgcggggaaagatcctaagaaaactggaggacctatatacaggagagtaaacggaaagtggatgagagaactcatcctttatgacaaagaagaaataaggcgaatctggcgccaagctaataatggtgacgatgcaacggctggtctgactcacatgatgatctggcattccaatttgaatgatgcaacttatcagaggacaagagctcttgttcgcaccggaatggatcccaggatgtgctctctgatgcaaggttcaactctccctaggaggtctggagccgcaggtgctgcagtcaaaggagttggaacaatggtgatggaattggtcaggatgatcaaacgtgggatcaatgatcggaacttctggaggggtgagaatggacgaaaaacaagaattgcttatgaaagaatgtgcaacattctcaaagggaaatttcaaactgctgcacaaaaagcaatgatggatcaagtgagagagagccggaacccagggaatgctgagttcgaagatctcacttttctagcacggtctgcactcatattgagagggtcggttgctcacaagtcctgcctgcctgcctgtgtgtatggacctgccgtagccagtgggtacgactttgaaagagagggatactctctagtcggaatagaccctttcagactgcttcaaaacagccaagtgtacagcctaatcagaccaaatgagaatccagcacacaagagtcaactggtgtggatggcatgccattctgccgcatttgaagatctaagagtattaagcttcatcaaagggacgaaggtgctcccaagagggaagctttccactagaggagttcaaattgcttccaatgaaaatatggagactatggaatcaagtacacttgaactgagaagcaggtactgggccataaggaccagaagtggaggaaacaccaatcaacagagggcatctgcgggccaaatcagcatacaacctacgttctcagtacagagaaatctcccttttgacagaacaaccattatggcagcattcaatgggaatacagagggaagaacatctgacatgaggaccgaaatcataaggatgatggaaagtgcaagaccagaagatgtgtctttccaggggcggggagtcttcgagctctcggacgaaaaggcagcgagcccgatcgtgccttcctttgacatgagtaatgaaggatcttatttcttcggagacaatgcagaggagtacgacaat

SEQ ID NO: 28 - protein, 498 aa, Influenza A virus
MASQGTKRSYEQMETDGERQNATEIRASVGKMIGGIGRFYIQMCTELKLSDYEGRLIQNSLTIERMVLSAFDERRNKYLEEHPSAGKDPKKTGGPIYRRVNGKWMRELILYDKEEIRRIWRQANNGDDATAGLTHMMIWHSNLNDATYQRTRALVRTGMDPRMCSLMQGSTLPRRSGAAGAAVKGVGTMVMELVRMIKRGINDRNFWRGENGRKTRIAYERMCNILKGKFQTAAQKAMMDQVRESRNPGNAEFEDLTFLARSALILRGSVAHKSCLPACVYGPAVASGYDFEREGYSLVGIDPFRLLQNSQVYSLIRPNENPAHKSQLVWMACHSAAFEDLRVLSFIKGTKVLPRGKLSTRGVQIASNENMETMESSTLELRSRYWAIRTRSGGNTNQQRASAGQISIQPTFSVQRNLPFDRTTIMAAFNGNTEGRTSDMRTEIIRMMESARPEDVSFQGRGVFELSDEKAASPIVPSFDMSNEGSYFFGDNAEEYDN

SEQ ID NO: 29 - DNA, 357 bp, Human immunodeficiency virus type 1
  CDS (1)..(357): Regulator of virus expression; codon optimized for human expression. Codon optimized for Homo sapiens.
atggccggcagaagcggcgacagcgacgaggagctgctgaaaaccgtgcggctcatcaagttcctgtaccagagcaaccctccacccagccccgagggcaccagacaggcccggagaaaccggaggaggcggtggagagagaggcagcggcagatcagaagcatcagcgagcggattctgagcacctacctgggcagaagcgccgagcccgtgcccctgcagctgccccccctggagagactgaccctggactgcaatgaggattgcggcaccagcggcacccagggcgtgggcagcccccagatcctggtggagagccctgccgtgctggagagcggcaccaaggaagatctgtga

SEQ ID NO: 30 - protein, 118 aa, Human immunodeficiency virus type 1
MAGRSGDSDEELLKTVRLIKFLYQSNPPPSPEGTRQARRNRRRRWRERQRQIRSISERILSTYLGRSAEPVPLQLPPLERLTLDCNEDCGTSGTQGVGSPQILVESPAVLESGTKEDL

SEQ ID NO: 31 - DNA, 357 bp, Human immunodeficiency virus type 1
  CDS (1)..(357): Codon optimized for Homo sapiens.
atggccggcagaagcggcgacagcgacgaggagctgctgaaaaccgtgcggctcatcaagttcctgtaccagagcaaccctccacccagccccgagggcaccagacaggcccggagaaaccggaggaggcggtggagagagaggcagcggcagatcagaagcatcagcgagcggattctgagcacctacctgggcagaagcgccgagcccgtgcccctgcagctgccccccctggagagagccaccctggactgcaatgaggattgcggcaccagcggcacccagggcgtgggcagcccccagatcctggtggagagccctgccgtgctggagagcggcaccaaggaagatctgtga

SEQ ID NO: 32 - protein, 118 aa, Human immunodeficiency virus type 1
MAGRSGDSDEELLKTVRLIKFLYQSNPPPSPEGTRQARRNRRRRWRERQRQIRSISERILSTYLGRSAEPVPLQLPPLERATLDCNEDCGTSGTQGVGSPQILVESPAVLESGTKEDL

SEQ ID NO: 33 - DNA, 309 bp, Human immunodeficiency virus type 1
  CDS (1)..(309): Transactivator; HIV-I consensus subtype B Tat. Codon optimized for Homo sapiens.
atggagcccgtggaccctagactggagccttggaagcaccccggcagccagcccaagaccgcctgcaccaactgctactgcaagaagtgctgcttccacgcccaggtgtgcttcatcaccaagggcctgggcatcagctacggccggaagaagaacaacctgagcaggagagccccccaggacagccagacccaccaggtgagcctgagcaagcagcctgccagccagcctagaggcgagcccaccggccccaaggagagcaagaagaaggtggagcgggagaccgagaccgatcccgtagatctgtga

SEQ ID NO: 34 - protein, 102 aa, Human immunodeficiency virus type 1
MEPVDPRLEPWKHPGSQPKTACTNCYCKKCCFHAQVCFITKGLGISYGRKKNNLSRRAPQDSQTHQVSLSKQPASQPRGEPTGPKESKKKVERETETDPVDL

SEQ ID NO: 35 - DNA, 309 bp, Human immunodeficiency virus type 1
  CDS (1)..(309): Transactivator; HIV-I consensus subtype B Tat. Codon optimized for Homo sapiens.
atggagcccgtggaccctagactggagccttggaagcaccccggcagccagcccaagaccgcctgcaccaactgctactgcaagaagtgctgcttccactgccaggtgtgcttcatcaccaagggcctgggcatcagctacggccggaagaagcggagacagaggcggagagccccccaggacagccagacccaccaggtgagcctgagcaagcagcctgccagccagcctagaggcgaccccaccggccccaaggagagcaagaagaaggtggagcgggagaccgagaccgatcccgtagatctgtga

SEQ ID NO: 36 - protein, 102 aa, Human immunodeficiency virus type 1
MEPVDPRLEPWKHPGSQPKTACTNCYCKKCCFHCQVCFITKGLGISYGRKKRRQRRRAPQDSQTHQVSLSKQPASQPRGDPTGPKESKKKVERETETDPVDL

(19) United States
(12) Patent Application Publication
(10) Pub. No.: US 2007/0087333 A1
(43) Pub. Date: Apr. 19, 2007

(54) Method to detect antigen-specific cytolytic activity

(75) Inventors: Robertus Antonius Gruters, Utrecht (NL); Carel Adriaan van Baalen, Zeewolde (NL); Gustaaf Frank Rimmelzwaan, Bergschenhoek (NL); Albertus Dominicus Marcellinus Erasmus Osterhaus, Bunnik (NL)

Correspondence Address: TRASK BRITT, P.O. BOX 2550, SALT LAKE CITY, UT, 84110, US

(73) Assignee: Erasmus Universiteit Rotterdam, Rotterdam (NL)

(21) Appl. No.: 11/506,418

(22) Filed: Aug. 18, 2006

Related U.S. Application Data
(63) Continuation of application No. PCT/NL05/00119, filed on Feb. 18, 2005.

(30) Foreign Application Priority Data
Feb. 20, 2004 (EP) 04075555.5

(51) Int. Cl. C12Q 1/70; C12Q 1/68; G01N 33/567

(52) U.S. Cl. 435/5; 435/6; 435/7.2

(57) ABSTRACT

The invention relates to a novel non-radioactive method to detect cytolytic activity that provides a measure of the existence and magnitude of an immune response against a particular antigen or immunogen. Provided is a method for detecting cytolytic activity of cells or a substance against a population of target cells, comprising the steps of providing target cells with a first nucleic acid sequence encoding a reporter molecule and a second nucleic acid sequence encoding an antigen of interest; co-culturing the target cells with a sample containing cells or a substance suspected of having cytolytic activity; and detecting the viability of target cells provided with the reporter molecule. Also provided are a kit and a nucleic acid for use in a method according to the invention.

### CROSS-REFERENCE TO RELATED APPLICATIONS

This application is a continuation of PCT International Patent Application No. PCT/NL2005/000119, filed on Feb. 18, 2005, designating the United States of America, and published, in English, as PCT International Publication No. WO 2005/080991 A1 on Sep. 1, 2005, which application claims priority to European Patent Application Serial No. 04075555.5, filed Feb. 20, 2004, the entire contents of each of which are hereby incorporated herein by this reference.

### TECHNICAL FIELD

The invention relates to a novel non-radioactive method to detect cytolytic activity against target cells expressing a specific antigen of choice. Cytotoxic T-lymphocyte (CTL) activity provides a measure of the existence and magnitude of a cell-mediated cytotoxic response against a particular antigen. Antibody-mediated cytotoxic activity quantifies the humoral immunity against the particular antigen.

### BACKGROUND

CTLs continuously survey cells from the body as a line of defense against aberrant behavior of these cells. This unwanted behavior includes the production of foreign proteins after infection by pathogens or after transformation into a new phenotype, with uncontrolled growth in cancer cells (but also the introduction of foreign cells into the body). CTLs are educated and selected to not recognize cells that express their normal phenotype and recognize foreign cells by their expression of unknown (non-self) protein fragments in the context of molecules of the major histocompatibility complex (MHC) at the cell surface. The MHC encodes polymorphic cell surface proteins (human leukocyte antigens (HLA)), which play a key role in the antigen-specific immune response. The MHC molecules are synthesized intracellularly and transported to the membrane after assembly with an antigenic epitope, usually a peptide derived from an intracellularly synthesized protein. The MHC-peptide complex is bound specifically by the T-cell receptor (TCR) via interactions at the atomic level, similar to antibody-antigen binding. Recognition of the specific target results in the organization of an immunological synapse. Recruitment of more TCR molecules into the immunological synapse continues until a threshold is reached. This results in the internalization of the TCR, together with fragments of the target cell, after which the CTL is activated. CTL activation typically results in the delivery of various signals to the target cells, including: i) secretion of granules containing granzymes and perforin, ii) synthesis of cytokines and/or chemokines, iii) cell signaling via membrane receptors, including Fas-FasL. CTL activation results in changes in the target cell, including a stop of protein synthesis, induction of DNA fragmentation as a part of apoptosis and leakage of the cell contents due to pores in the membrane. As a result, the target cell will die, preventing further production of pathogens or proliferation of cancer cells.

Various assays have been developed over time to study the processes that follow CTL-target interactions. In the past three decades, the ⁵¹Cr-release assay has been used to quantify antigen-specific cell-mediated cytotoxicity activity (Brunner et al. (1968), Immunology 14:181-196). In this assay, target cells labeled with radioactive isotope ⁵¹Cr are incubated with CTL cells for four to six hours. Target cell death is then measured by detecting radioactivity released into the culture supernatant. Although relatively reproducible and simple, this assay has numerous disadvantages (Doherty and Christensen (2000), Annu. Rev. Immunol. 18:561-592).

First, bulk cell-mediated cytotoxicity activity is measured using “lytic unit” calculations that do not quantify target cell death at the single-cell level.

Second, CTL-mediated killing of primary host target cells often cannot be studied directly, as only certain types of cells, primarily immortalized cell lines, can be efficiently labeled with ⁵¹Cr (Nociari et al. (1998), J. Immunol. Meth. 213:157-167).

Third, target cell death is measured at the end point of the entire process and thus provides little information about the kinetic interaction of effectors and targets at the molecular and cellular levels.

Fourth, the radioactive conventional assay using ⁵¹Cr results in a very high background (noise) signal due to a large amount of spontaneous non-specific cell death or other types of release of the isotope from the target cells. Thus, the amount of released radioactivity is not a direct measure of cell death but rather a measure of increased membrane permeability and spontaneous release of the isotope from the loaded cells due to processes other than the cellular cytotoxicity brought about by the CTLs.

Fifth, loading the selected target cells with the isotope is often very heterogeneous. Consequently, the conventional chromium release assay has difficulty in detecting definite but less potent cytotoxic effects, i.e., it is difficult to distinguish a signal caused by cell-mediated cytotoxic activity from the assay's background radioactivity. Furthermore, measurement of ⁵¹Cr release does not permit monitoring the physiology or fate of effector cells as they initiate and execute the killing process.

Finally, radioactive materials require special licensing and handling, which substantially increases cost and complexity of the assay.

More recently developed immunologic methods, including major histocompatibility complex (MHC)-tetramers, intracellular cytokine detection and Elispot assays, have greatly improved sensitivity to enumerate antigen-specific T-cells. The Elispot assay measures cytokine production by CTLs after activation (see, for example, F. H. Rininsland et al. (2000), J. Immunol. Methods 240:143-155). The produced cytokines are captured by specific antibodies bound to a support and revealed by a second antibody coupled to an enzyme that precipitates a substrate, resulting in a visible spot. Intracellular staining assays similarly assess cytokine production, but the capture of cytokines is done intracellularly, after blocking export of the cytokines. Read out is generally performed by FACS analysis. The disadvantages of this assay include the interpretation and reproducibility of the results.

Tetramer staining involves the use of solubilized MHC molecules, assembled into a tetramer presenting the specific peptide recognized by a (known) CTL population (J. D. Altman et al. (1996), Science 274:94-96). The assay is very sensitive in detecting CTLs, but has the disadvantage that only a predetermined CTL population can be detected and it does not measure their activity or capacity to kill. Furthermore, tetramer staining is very expensive.

Recently, CD107a/b staining was described (M. R. Betts et al. (2003), J. Immunol. Methods 281(1-2):65-78). The CD107a/b membrane molecules are normally resident in the secretory granules inside the CTLs and are only expressed at the surface transiently after CTL activation, when the granules have been secreted. During this period, specific antibodies can detect the presence of cell surface CD107a/b, thus finding the “smoking gun” of the lethal hit that the CTLs delivered.

Yet another way to determine CTL activity involves the use of a fluorescent lipophilic dye (e.g., PKH-26) that stably integrates into cell membranes and can be detected by flow cytometry (Fischer et al. (2002), J. Immun. Methods 259(−1):159-169; Hudrisier et al. (2001), J. Immun. 3645-3649). Following co-incubation of dye-labeled target cells with non-labeled CTLs, capture of target cell membranes by CTLs can be measured as a decrease in target cell fluorescence. Capture of labeled target cell membranes by CTLs can also be determined as an increase in CTL fluorescence. However, due to the rapid degradation of labeled target cell membrane acquired by the CTLs, the drawback of monitoring dye uptake by CTLs is the very short observation window (one-half to two hours).

Recently, Tomaru et al. reported the detection of CTL activity by measuring the acquisition of peptide-HLA2-GFP complexes by CTL from target cells expressing a HLA2-GFP construct (Nature Medicine, 2003, Vol. 9, pp. 469-475). In contrast to the dye system, this system allows selectively measuring antigen-specific CTL activity. A major limitation of this system is that it is always restricted to the HLA type chosen. For example, it would not be possible to apply this method in a clinical setting wherein various patients' samples, with various HLA types, are to be analyzed. Moreover, it does not measure the actual cell killing activity of CTLs. Thus, a major drawback of these newer methods is that they do not assess the cytolytic function of antigen-specific CTLs (Altman et al. (1996), Science 274:94-96 (1996); erratum: 280:1821 (1998); Butz and Bevan (1998), Immunity 8:167-175; Maino and Picker (1998), Cytometry 34:207-215). Given the emerging data indicating that antigen-specific CD8+T cells may be present in certain chronic infections or malignancies but blocked from their ability to lyse target cells, assays that accurately measure the cell-killing activity of CTLs, preferably at the single-cell level, are needed (Appay et al. (2000), J. Exp. Med. 192:63-75; Lee et al. (1999), Nature Med. 5:677-685; Zajac et al. (1998), J. Exp. Med. 188:2205-2213).

Besides the choice of how to detect CTL activity, the preparation of target cells is an important determinant regarding the specificity and sensitivity of the CTL activity assay. CTL assays can be performed with peptide-loaded target cells. A known peptide, or a set of overlapping peptides, is added extracellularly to the cells to occupy via exchange the cleft of specific MHC molecules, after which the MHC-peptide complex can be recognized by CTLs. Different concentrations of peptides may, however, induce different CTL responses.

Alternatively, target cells can be infected with a recombinant virus vector (usually vaccinia) that encodes a protein or peptide of interest. This allows for a more physiological intracellular synthesis of the MHC-peptide complex. However, the disadvantage of this technique lies in the rapid lysis of the target cells by the vaccinia vector itself, which severely limits the time for manipulation and observation.

### DISCLOSURE OF THE INVENTION

The present invention solves the problems of the known CTL assays. Provided is a method for detecting cytolytic activity of cells or a substance against a population of target cells, comprising providing target cells with a first nucleic acid sequence encoding a reporter molecule and a second nucleic acid sequence encoding an antigen of interest; co-culturing the target cells with a sample containing cells or a substance suspected of having cytolytic activity; and detecting the viability of target cells provided with the reporter molecule, wherein a loss of target cell viability is indicative of cytolytic activity. The general principle of the assay according to the invention is schematically depicted in FIG. 1. Briefly, cytotoxicity is quantified by assessing the elimination of viable target cells that express both an antigen of interest as well as a fluorescent reporter molecule (e.g., generated by transfecting recombinant DNA vectors encoding antigen-fluorescent fusion proteins). Elimination of viable antigen-reporter molecule-expressing target cells (T) by cytotoxic effector cells (E) can be detected by any device or method that is designed to detect reporter gene expression; for instance, GFP-expressing cells can be detected by flow cytometry (FIG. 1B). See legend of FIG. 1 for further explanation.

Using in vitro-generated antigen-specific cytotoxic T-lymphocytes or ex vivo PBMC, it was found that an assay based on a method provided herein is sensitive, performs very well compared with the standard ⁵¹Cr release assay (see FIG. 4), and is easy to handle. The method disclosed is, of course, also suitable to detect cytolytic activity of cells other than CTLs, for example, antigen-specific CD4+ T-helper (Th) cells, and natural killer (NK) cells. In one aspect of the invention, a method is provided to detect antibody-induced killing of a target cell, including antibody-dependent cell-mediated cytotoxicity (ADCC) and complement-dependent cytotoxicity (CDC).

ADCC involves the attachment of an antigen-specific antibody to a target cell and the subsequent destruction of the target cell by immunocompetent cells. Fc receptors on immunocompetent cells recognize the Fc portion of antibodies adhering to surface antigens. Most commonly, the effector cell of ADCC is a natural killer (NK) cell. Following recognition and attachment via its Fc receptors, the NK cell can destroy the target cell through release of granules containing perforin and granzyme B and/or activation of the FAS/FAS ligand apoptosis system in the target cell. Perforin molecules make holes or pores in the cell membrane, disrupting the osmotic barrier and killing the cell via osmotic lysis.

Complement-dependent cell-mediated cytotoxicity involves the recognition and attachment of complement-fixing antibodies to a specific surface antigen followed by complement activation. Sequential activation of the components of the complement system ultimately lead to the formation of the membrane attack complex (MAC) that forms transmembrane pores that disrupt the osmotic barrier of the membrane and lead to osmotic lysis. The MACS function similarly to the perforin molecules released by cytolytic T-cells and NK cells, killing cells by osmotic lysis.

In contrast to indirect evaluation of cytotoxicity using radioactive assays, an assay according to the invention is based on the quantitative and qualitative (flow cytometric) analysis of target cell death on a single cell level. Moreover, due to the ability to selectively analyze the (loss of) viability of the subpopulation of antigen-expressing target cells, the sensitivity of the method provided is higher than that of conventional methods comprising non-specific labeling of all target cells. In addition, the present invention makes it possible to detect activity of CTL without knowing the specificity and HLA-restriction of the CTL. Importantly, and in contrast to traditional CTL assays, a method of the invention is highly suitable for monitoring CTL functions in a routine (e.g., clinical laboratory or research) setting that requires simple and reproducible assay techniques.

A method of the invention involving the use of target cells that have been provided with both an exogenous antigen of interest and a reporter molecule is not known in the art. Fischer et al. (2002), J. Immun. Methods 259(−1):159-169, describes a flow cytometric assay for the determination of cytotoxic T-lymphocyte activity using non-transfected tumor cells comprising endogenous tumor antigens, which cells are stained with lipophilic dye. Flügel et al. (1999), Int. J. of Dev. Neuroscience, pp. 547-556, discloses a non-radioactive cytotoxicity assay for GFP-transduced tumor cells. Also here, the target cells are not provided with an exogenous antigen of interest. Flierger et al. (1995), J. of Immunol. Methods, vol. 180, pp. 1-13, and Mattis et al. (1997), J. of Immunol. Methods, vol. 204, pp. 135-142, describe membrane-uptake assays using either PKH-26 labelled or DiO₁₈(3)-labeled target cells. The target cells are not provided with exogenous antigen of interest for endogenous expression.

The term “reporter molecule,” as used herein, refers to a molecule (e.g., a polypeptide or protein fragment) that comprises a detectable label, for example, a fluorescent label or a chemical dye, or to a molecule that can be detected using a detectable probe that specifically binds to the molecule. In one embodiment, a reporter molecule is a fluorescent polypeptide. In another embodiment, the reporter molecule is a cell surface marker that can be detected using a fluorescently labeled (monoclonal) antibody.

In a further aspect of the invention, target cells are provided with a reporter molecule and an antigen of interest, wherein the reporter molecule is stably associated with plasma membrane of the target cell. A reporter molecule (e.g., GFP) of the invention may also be targeted to the plasma membrane of target cells by procedures well known in the art. These include providing the reporter molecule with a fatty acyl chain (e.g., palmitate or myristate) or with the membrane-anchoring domain of a known membrane-associated protein, such as amino acids 1 through 10 of p561ck or the C-terminal CaaX motif required for membrane association of Ras and Rho GTPases. Similar to the PKH-uptake assay, CTL activity can then also be assessed by determining the uptake of the target cell membrane comprising the reporter molecule. In contrast to the PKH assay wherein all target cells are labeled, only the plasma membranes of target cells comprising the antigen of interest are labeled with a reporter molecule.

A fluorescent reporter molecule or a fluorescent antibody bound to a reporter molecule allows for the detection of labeled target cells by various standard fluorescence detection techniques known in the art, including fluorescence-activated cell sorting (FACS), also referred to as flow cytometry, immunofluorescence (IF), or a fluorometer, e.g., a 96-well fluorescence reader. FACS analysis is highly suitable to determine viability of individual cells, in particular, that of non-adherent cells, as the forward scatter (FSC) and side scatter (SSC) characteristics of viable cells differ from those of non-viable cells, and several fluorescent dyes for viable-dead discrimination have been successfully used. A suitable fluorescent reporter molecule is GFP (green fluorescent protein) and spectral variants thereof, such as YFP (yellow fluorescent protein) and CFP (cyan fluorescent protein). GFP, a 27-kD polypeptide, is intrinsically fluorescent, thus, it does not need substrates or co-factors to produce a green emission when appropriately excited, e.g., with UV light or 488 nm laser light. A GFP-modified version, with the alterations Ser 65 to Thr and Phe 64 to Leu, was named EGFP (enhanced green fluorescent protein; Cormack et al., 1996). EGFP produces fluorescence 35 times more intense than wild-type GFP and has a better solubility, as well as faster folding and chromophore maturation (Kain and Ma, 1999). In one embodiment, enhanced GFP or an enhanced spectral variant thereof (e.g., ECFP or EYFP), or any other fluorescent protein including (but not exclusively) hcRed or dsRed, is used in a method of the invention. In another embodiment, a reporter molecule is a cell surface (e.g., transmembrane) protein that is detected using a fluorescent antibody that binds to the cell surface protein.

Various types of cells may be used as target cells in a CTL assay according to the invention. Target cells can be primary cells, such as peripheral blood mononuclear cells (PBMC) or cells from a cell line. Cell lines are cells that have been extracted from human or animal tissue or blood and capable of growing and replicating continuously outside the living organism, for instance, Epstein-Barr virus transformed B-lymphoblastoid cell lines (B-LCL).

For a skilled person, it will be clear that by using target cells expressing an antigen of interest, a method as provided permits the detection of CTL activities against various types of antigens. An antigen of interest can be selected from the group consisting of a viral, bacterial, parasitic or tumor antigen. Viral antigens include antigens from Influenza virus, Herpes viruses, human immunodeficiency virus (HIV), hepatitis A virus (HAV) hepatitis B virus (HBV), hepatitis C virus (HCV), measles (Rubeola) virus, respiratory syncytial virus (RSV), human metapneumovirus (hMPV), severe acute respiratory syndrome (SARS) virus, Corona virus, and the like. Also included are viral antigens that have yet to be identified as well as fragments, epitopes and any and all modifications thereof, such as amino acid substitutions, deletions, additions, carbohydrate modifications, and the like.

In one embodiment, target cells are provided with an influenza viral nucleoprotein (NP) or matrix protein and are used in a method provided herein to determine influenza-specific CTL activity. In another embodiment, the antigen of interest is an HIV antigen. Preferred antigens include Env, Tat, Rev, Gag, Nef and Vpr of HIV. These antigens can be cloned in frame with a fluorescent reporter molecule (see also Example 2). In yet another embodiment, an antigen of interest is an antigen from Malaria parasite (Plasmodium falciparum) or a Mycobacterium tuberculosis antigen.

The term “tumor antigen” as used herein includes both tumor-associated antigens (TAAs) and tumor-specific antigens (TSAs). A tumor-associated antigen refers to an antigen that is expressed on the surface of a tumor cell in higher amounts than is observed on normal cells or to an antigen that is expressed on normal cells during fetal development. A tumor-specific antigen is an antigen that is unique to tumor cells and is not expressed on normal cells. Tumor antigens that can be used include: i) cancer-testis antigens (CTA), expressed in tumors of various histology but not in normal tissues, other than testis and placenta, such as, for example, MAGE, GAGE, SSX SART-1, BAGE, NY-ESO-1, XAGE-1, TRAG-3 and SAGE, some of which represent multiple families (C. Traversari (1999), Minerva Biotech., 11:243-253); ii) differentiation-specific antigens, expressed in normal and neoplastic melanocytes, such as, for example, tyrosinase, Melan-A/MART-1, gp100/Pmel17, TRP-1/gp75, TRP-2 (C. Traversari (1999), Minerva Biotech., 11:243-253); iii) antigens over-expressed in malignant tissues of different histology but also present in their benign counterpart, for example, PRAME (H. Ikeda et al. (1997), Immunity, 6:199-208), HER-2/neu (C. Traversari (1999), Minerva Biotech., 11:243-253), CEA, MUC-1 (G. M. Monges et al. (1999), Am. J. Clin. Pathol. 112:635-640), alpha-fetoprotein (W. S. Meng et al. (2001), Mol. Immunol., 37:943-950; and iv) antigens derived from point mutations of genes encoding ubiquitously expressed proteins, such as MUM-1, α-catenin, HLA-A2, CDK4, and caspase 8 (C. Traversari (1999), Minerva Biotech., 11:243-253).

In a further embodiment of the invention, target cells are used that are provided with a tumor antigen that is derived from a tumor virus, i.e., a virus that uses DNA to code its genome and causes tumors in mammals, for example, an antigen derived from human papillomavirus (HPV).

A method as provided herein typically starts with the provision of a target cell population wherein at least part of the population is provided with a first nucleic acid sequence encoding an antigen of interest and a second nucleic acid sequence encoding a reporter molecule. Subsequently, these target cells are allowed to translate the nucleic acids encoding the antigen and the reporter molecule. Following translation, molecular chaperones help to protect the incompletely folded polypeptide chains from aggregating. Even after the folding process is complete, however, a protein can subsequently experience conditions under which it unfolds, at least partially, and then it is again prone to aggregation. Proteins in “non-functionally” (unfolded/partially) folded configurations are more likely to be degraded. Degraded polypeptides can assemble intracellularly with the MHC complex and are transported as an MHC-peptide complex to the cell surface. The percentage of non-functionally folded polypeptide ranges between approximately 5 and 50%, depending on the polypeptide. Thus, during normal cellular protein turnover, at least part of the expressed antigen is proteolytically processed to generate one or more antigenic epitopes that are displayed at the surface of the target cells, allowing for recognition of the antigenic epitope by CTLs. Whereas the reporter molecule will also be processed to a certain extent, the proportion that remains intact will be sufficient to identify the target cells.

Target cells can be provided with the nucleic acid sequences by known procedures, typically involving the introduction of an expression plasmid (also known as vectors) carrying the sequences into the cells by a process called transfection. Transfection refers to the introduction of foreign DNA into a recipient host cell. The foreign DNA may or may not subsequently integrate into the chromosomal DNA of the recipient cell before transcription and translation occur. Transfection is readily accomplished via a variety of methods known in the art, including DNA precipitation with calcium ions, electroporation and cationic-lipid-based transfection methods. Electroporation is the reversible creation of small holes in the outer membrane of cells as a result of high electric fields affecting the cells. While the cells are porous, fluids and substances including foreign DNA can enter into the cytoplasm.

In a preferred embodiment, target cells are provided with nucleic acid encoding an antigen and a reporter molecule (e.g., GFP) using Nucleofector™ technology. Based on electroporation, the Nucleofector™ concept uses a combination of electrical parameters and cell-type-specific buffer solutions. The Nucleofector™ technology is unique in its ability to transfer DNA directly into the nucleus of a cell. Thus, cells with limited ability to divide, such as primary cells and hard-to-transfect cell lines, are made accessible for efficient gene transfer (see www.amaxa.com). The transfection efficiency of primary target cells using nucleofection can reach >50%. Alternatively, target cells are provided with an antigen of interest and a reporter molecule using a viral delivery system. This virus delivery system may be the pathogen of interest containing a reporter gene, e.g., HIV-GFP or Influenza virus-GFP.

According to the invention, only target cells provided with the reporter molecule are assumed to display the antigenic epitope. Thus, co-expression of antigen and reporter in the same cells (it does not matter whether they are fused or not) is important. Antigen and reporter can be expressed in the same cell using a variety of strategies, including: i) two separate vectors (as long as all cells expressing reporter gene also express antigen); ii) one vector with multiple promoters, multicistronic niRNAs (e.g., use vector with an IRES) etc.; and iii) recombinant virus under study.

In an embodiment using separate plasmids, the antigen-expressing plasmid may drive the expression of reporter-expressing plasmid (e.g., if former expresses Tat and latter has a TAR element). In case separate vectors are used, the optimal ratio of the vectors can be optimized to ensure that all cells expressing the reporter molecule also express the antigen.

In another embodiment, nucleic acid sequences encoding an antigen and a reporter molecule are provided to the target cell simultaneously, for example, by nucleofection of a single expression vector comprising both sequences. Such a vector may comprise two separate promoters to express each of the reporter molecule and the antigen or it may contain an (Internal Ribosome Entry Site) IRES. The use of IRES allows the co-expression of multiple molecules from a single mRNA. Alternatively, the antigen (epitope or protein) may be cloned in frame with the Open Reading Frame (ORF) of the reporter molecule, e.g., GFP, such that the nucleic acid sequences are expressed in the target cell as one fusion protein comprising the antigen (Ag) and the reporter molecule.

Various expression vectors suitable for use in a method of the invention are commercially available, for example, the C- or N-Terminal Fluorescent Protein Vectors from BD Clontech (BD, Franklin Lakes, N.J., USA). These vectors comprising a CMV promoter allow the expression of fluorescent fusion proteins in mammalian cells. A nucleic acid sequence encoding an antigen of interest inserted into the multiple cloning site (MCS) of these vectors will be expressed as a fusion to either the C- or N-terminus of a fluorescent reporter protein, such as, DsRed2, ECFP, EGFP, EYFP, or HcRed1. In a preferred embodiment, the antigen of interest is cloned N-terminally in frame with the reporter molecule that can stably associate with the plasma membrane, for example, resulting in an antigen-GFP fusion (Ag-GFP) protein. Herewith, the invention provides an expression vector comprising a first nucleic acid sequence encoding a reporter molecule, a second nucleic acid sequence encoding an antigen of interest and the regulatory elements needed to express the sequences in a target cell, wherein the antigen and the reporter molecule are expressed as a fusion protein, preferably wherein the antigen is fused to the N-terminus of the reporter molecule. Preferably, the vector encodes a viral antigen fused to the N-terminus of GFP. More preferably, the vector encodes an antigen derived from an HIV protein, such as Gag, Tat, Rev, Vpr or Nef, or an antigen derived from an influenza protein, such as a nucleoprotein or matrixprotein (see Example 2 and FIG. 7).

In a further embodiment, a target cell can be infected with a recombinant pathogen expressing a reporter molecule, such as GFP. For evaluating the CTL response to a virus, one can challenge a target cell with a recombinant virus that expresses a reporter. In one embodiment, CTL activity against HIV is detected using target cells that have been infected with HIV delta Env pseudotyped with VSV-G in which GFP is expressed instead of Env or Nef. Using such a viral delivery approach, 90 to 100% of the target cells can be provided with the antigen of interest and the reporter molecule. In yet a further embodiment, a target cell is infected with (wild-type) pathogen and the target cells are subsequently detected using a (labeled) antibody directed against a cell surface marker of that pathogen (e.g., infect target cells with HIV and detect antigen-presenting target cells with anti-gp120 Mab).

Target cells that have been successfully provided with a reporter molecule can be identified by various means known in the art, as detailed before. The presence of the antigen can be verified by double staining the cells with an antigen-specific probe (for instance, an antibody) that is conjugated to a distinguishable label, e.g., the red dye phycoerythrine (PE) in case GFP is used as reporter molecule.

In a next step, the target cells are co-cultured or co-incubated with cells or a substance suspected of having cytolytic activity (e.g., CTLs, CD4, NK, ADCC), antibody plus complement. The cells can be present in a sample obtained from an animal, preferably a human. It may be a clinical sample, for example, a sample obtained from a (human) patient suspected of having cancer, an infectious disease, or from a vaccinated subject. Of course, a method of the invention may also be used in a research setting, e.g., to monitor the function of CTLs or screen a test agent for the ability to induce an antigen-specific CTL response in an animal, including humans and laboratory animals. The term “co-cultured” as used herein refers to placing cytolytic cells or substance (antibody) and target cells into a buffer and/or medium wherein the cells or substance are capable of interacting (e.g., inducing a cytotoxic response). In certain embodiments, co-culturing may involve heating, warming, or maintaining the cells at a particular temperature and/or passaging of the cells. In conventional CTL assays, such as the ⁵¹Cr assay, a considerable excess of CTLs relative to the number of target cells is required to obtain a detectable amount of target cell lysis. Typically, an effector to target ratio (E:T) ranging from 10 to 1 is used. In contrast, target cell lysis according to a method provided herein can be detected at surprisingly low effector:target ratios, e.g., as low as 0.03 after a four-hour assay. In the ⁵¹Cr-release assay, similar results can only be achieved if 100% of the ⁵¹Cr-loaded cells express the correct MHC-epitope complexes at their cell surface (see FIG. 4), e.g., after loading with saturating amounts of peptide.

Known CTL assays have a rather limited observation window, i.e., the time period following initiation of the co-culturing that can be used to determine CTL activity. Depending on the assay, the conventional observation window is two to five hours (⁵¹Cr assay, CD 107 staining); six to twelve hours (Elispot) or only one-half to two hours (PKH uptake assay). Surprisingly, according to a method of the invention, co-cultures can be followed for various time periods (2 to 72 hours or longer) to determine CTL-mediated lysis of target cells. Thus, a method as provided herein has a much wider observation window than any of the conventional CTL assays, allowing for increased sensitivity.

Following co-culturing, CTL-mediated lysis (loss of viability) of the target cells is determined. Specific target cell lysis can be determined in various ways. In one embodiment, it is determined by measuring a decrease in the fraction of viable target cells comprising a reporter molecule. For example, specific lysis can be measured using flow cytometry by comparing the fraction of dead events among GFP-expressing target cells that have been cultured with and without CTL. An increase in non-viable GFP-positive target cells that have been co-incubated with CTL is indicative of CTL-specific lysis. Alternatively, specific lysis can be determined from the decrease in the number of GFP-positive events between target cell cultures with and without CTL.

There are several methods that can be used to quantitate viability of cells. These methods typically use so-called viability dyes (e.g., propidium iodide (PI), 7-Amino Actinomycin D (7-AAD)) that do not enter cells with intact cell membranes or active cell metabolism. This cyanine dye is suitable for use with an Argon laser. Cells with damaged plasma membranes or with impaired/no cell metabolism are unable to prevent the dye from entering the cell. Once inside the cell, the dyes bind to intracellular structures producing highly fluorescent adducts that identify the cells as “non-viable.” In a preferred embodiment, a nucleic acid stain is used as viability dye, such as TO-PRO-3 iodide (TP3). TP3 is a nucleic acid stain that absorbs and emits in the far red region (643/661 nm, FL4) and is suitable for use as a viability stain (dead cells take up TP-3; see FIG. 1B). TP3 and other suitable viability dyes are commercially available, for example, from Molecular Probes, Eugene, OR, USA (www.molecularprobes.com). Other methods that can be used to assess viability involve detection of active cell metabolism that can result in the conversion of a non-fluorescent substrate into a highly fluorescent product (e.g., fluorescein diacetate). Furthermore, dead cells can be discriminated by FACS analysis from viable cells based on their light scatter characteristics. In one embodiment of the invention, non-viable target cells are identified by the uptake of a viability dye in combination with altered light scatter characteristics (see FIG. 1).

A method of the invention comprises detection of target cells in a mixture of target cells and CTLs (effector cells). Target cells can be distinguished from effector cells by the exclusive presence of the reporter molecule in target cells and not in the effector cells. However, it may be advantageous to use an additional probe to distinguish between target cells and effector cells, for example, a detection probe capable of detecting a cell surface marker that is specific for either target cells or effector cells. Preferably, the detection probe is conjugated to a detectable label, more preferably a fluorescent label, to allow for detection of cells expressing the surface marker by flow cytometry. In one embodiment, following co-culturing of target cells with CTLs for a certain period, the mixture of target cells and CTLs is contacted with PE-conjugated anti-CD8 mAb (commercially available from DAKO, Glostrup, Denmark), a fluorescent probe capable of recognizing CD8 expressed on CTLs. Subsequently, target cells can be selectively analyzed using flow cytometry by gating out the CD8-positive cells, representing the CTLs.

CTL research has gained much interest in recent years since the pivotal role of CTLs in the control of infectious disease and cancer became clear. Their importance has been shown in the clearance of acute infections, the control of chronic disease and in the protection against (re-)infections after convalescence or vaccination. Similarly, CTLs have been found responsible for the control of cancer cell growth and the elimination of cancer cells. The novel assay provided herein is of use for the screening of both naturally acquired cellular immunity and vaccine-induced cellular immunity. The assay can also be used to monitor the development of cellular immune responses during chronic infection and cancer. The monitoring of these processes becomes rapidly more important with growing numbers of vaccination programs aimed at the induction of cellular immunity. Monitoring may prove cost effective in preventing obsolete treatment, e.g., in HIV infection and cancer.

With accumulating evidence that virus-specific CTLs are important in containing the spread of HIV-1 in infected individuals, a consensus has emerged that an HIV-1 vaccine should stimulate the generation of CTLs. This requirement has posed a number of challenges for HIV-1 vaccine development. A safe vaccine approach that induces high frequency, durable HIV-1-specific CTL responses has proven elusive. However, because traditional methods for measuring target cell lysis are cumbersome and difficult to quantify, monitoring the efficiency of vaccine-elicited HIV-1-specific CTL generation has been problematic. The invention now provides a quantitative and highly sensitive assay to detect an HIV-specific CTL response, which complements or even replaces traditional killing assays for monitoring HIV-1 vaccine trials in non-human primates and in humans. Thus, a method provided herein is advantageously used in studies directed at HIV-specific CTLs in various stages of disease. Such studies can provide important insights into AIDS pathogenesis and ultimately may lead to development of effective vaccine strategies.

In another aspect, this invention provides a method of screening a test agent for the ability to induce in a mammal cytolytic activity, e.g., a class I-restricted CTL response, directed against a particular antigen. The method typically involves administering to a mammal a test agent, obtaining effector cells (CTLs) from the mammal, and measuring cytotoxic activity of the CTLs against target cells displaying the antigenic epitope, where the cytotoxic activity is measured using any of the methods and/or indicators described herein, where cytotoxic activity of the effector cell against the target cell is an indicator that the test agent induces a class I-restricted CTL response directed against the antigen. For animal studies, Ag-GFP-expressing cells may be adoptively transferred to assess in vivo cytotoxic activity. See Rubio et al., Nat. Med. 9:1377-1382, and references therein, for examples with peptide-pulsed and tumor target cells.

This invention also provides a method of optimizing an antigen for use in a vaccine. The method typically involves providing a plurality of antigens that are candidates for the vaccine, screening the antigens using any of the methods described herein, and selecting an antigen that induces a class I-restricted CTL response directed against the antigen.

Also provided is a method of testing a mammal to determine if the mammal retains immunity from a previous vaccination, immunization or disease exposure. The method typically involves obtaining PBMC (e.g., containing CD8+cytotoxic T-lymphocytes) from the mammal and measuring cytotoxic activity of the CTLs against target cells displaying an antigenic epitope that is a target of an immune response induced by the vaccination, immunization, or disease exposure, where the cytotoxic activity is measured using any of the methods described herein, where cytotoxic activity of the CTLs against the target cells is an indicator that the animal retains immunity from the vaccination, immunization, or disease exposure.

Furthermore, the invention provides a kit of parts for use in a method according to the invention. Such a kit comprises an expression vector, preferably a eukaryotic expression vector, comprising a first nucleic acid sequence encoding an antigen of interest and a second nucleic acid sequence encoding a reporter molecule that can stably associate with the plasma membrane of a target cell (e.g., myristoylated GFP), and means for transfecting target cells with the expression vector. As mentioned above, the use of a reporter molecule that can be targeted to the plasma membrane has the advantage that, similar to the conventional PKH-uptake assay, CTL activity can also be assessed by determining the uptake of the target cell membrane comprising the reporter molecule. However, unlike the PKH assay wherein all target cells are labeled, only the plasma membranes of target cells comprising the antigen of interest are labeled with a reporter molecule.

Alternatively, a membrane protein can be used as an antigen for a specific antibody or a receptor for a specific ligand, where the antibody or the ligand are coupled to a reporter molecule, preferably a fluorescent group. Via this indirect procedure, Ag-expressing cells can be identified.

The first and second nucleic acid sequences may be present on the same or on separate expression vectors. In one embodiment, a kit comprises a vector comprising a nucleic acid sequence encoding a fusion of an antigen and a membrane-targeted reporter molecule. In another embodiment, a kit comprises more than one expression vector, one of which encodes the antigen of interest and the other one encodes the reporter molecule. In yet another embodiment, a kit comprises a vector comprising a first nucleic acid sequence encoding a reporter molecule and a multiple cloning site, which allows for the insertion of a second nucleic acid sequence encoding an antigen of interest. A kit according to the invention may further comprise at least one detectable probe capable of recognizing a cell surface marker that is specific for target cells or for CTLs (e.g., anti-CD8 mAb). Still further, a kit of the invention may comprise a viability dye to allow detection of dead target cells that have lost their membrane integrity. Preferably, a kit comprises a viability dye that stains the cellular DNA, for instance, TP3.

### BRIEF DESCRIPTION OF THE FIGURES

FIG. 1: Principles of the FATT-CTL assay. Panel A: Example of a procedure for the generation of fluorescent-antigen-transfected target cells. Antigen expression (in this example, the antigen is fused to the reporter) can be assessed using FACS analysis. Panel B: Elimination of viable antigen-reporter molecule-expressing target cells (T) by effectors cells (E) can be detected, for example, by flow cytometry. VG=viable GFP+ cells; DG=dead GFP+ target cells; % DG=percentage dead cells among the GFP+ cells; +E and −E refer to cultures with and without effector cells, respectively. Formula 1 can be used to calculate cell-mediated target cell elimination if the total number of GFP+ cells does not significantly change during the co-culture period. If incubation periods are long and dying target cells are largely disintegrated, specific target cell elimination can be expressed using formula 2.

FIG. 2: Construction of plasmid DNA vectors for the expression of antigen-fluorescent protein fusion proteins. Line A, construct of HIV genes encoding Rev, Tat, Gag and Nef were codon-optimized subtype B consensus sequence; Line B, multiple cloning site of N1 Living Colors™ vectors; Line C, spacer for creating in frame cloning site for the influenza genes; Line D, influenza virus genes encoding NP strain A/NL/18/94 (NP01), NP strain A/HK/2/68 (NP02), NP strain A/PR/8/34 (NP03), M1 strain A/NL/18/94 (M1).

FIG. 3: Antigen-specific killing of fluorescent-antigen transfected BLCL cells and PBMC by cloned CTL populations. Section A, GFP- and TP3-fluorescence intensities of pRev-GFP- (upper panels) or pTat-GFP-nucleofected (lower panels) B157 cells that had been co-cultured with or without cells of a Rev-specific CTL clone at the indicated E/T ratios for four hours. MFI of control GFP- events was ˜4. Numbers of viable and dead GFP+ events detected during a fixed acquisition period of constant flow rate are indicated. The percentage dead GFP+ events is shown in parentheses. Section B, left panel: percentage CTL-mediated lysis using values shown in Section A. Initial E/T ratios were calculated from numbers of CD8+and GFP+ events detected in cultures containing effector or target cells only, at t=0 hour. Right panel: antigen-specific lysis of pNPO-GFP-nucleofected PBMC by cells of influenza virus NP-specific CTL clone TCC-C10.

FIG. 4: Comparison between ⁵¹Cr-release and FATT-CTL assays. B157 cells were nucleofected with pRev-GFP or pTat-GFP and following overnight incubation half of the cells were labeled with ⁵¹Cr and used as target cells in a standard four-hour ⁵¹Cr-release assay. The other half was tested in a four-hour FATT-CTL assay. Target cells were co-cultured with Rev-specific CTL at indicated E/T ratios and percentage-specific lysis were determined as described in the methods section. T*: Calculation of initial E/T ratio in the FATT-CTL assay included GFP+ and GFP-target cells to allow direct comparison between the two assays.

FIG. 5: CTL-mediated killing of target cells expressing recombinant influenza virus NP- or M1-GFP proteins. B3180 cells were nucleofected with pNP01-GFP, pNP02-GFP, pNP03-GFP or pM1-GFP. The next day, these cells were co-cultured for three hours with or without TCC1.7, TCC-C10, TCC3180 and TCCM1/A2 cells at CD8+-to-GFP+ cell ratios of 10, 10, 5, and 2, respectively. CTL-mediated target cell death was determined as described in the methods section. *Functional avidity: EC50 value (nM) of the CTL clones for the epitope variants, as determined in a ⁵¹Cr-release assay [2].

FIG. 6: Ex vivo antigen-specific PBMC-mediated elimination of HIV-1 Gag-GFP- or Nef-GFP-expressing lymphocytes. PBMC obtained from four HIV-1 seropositive individuals were nucleofected with pEGFP-N1, pGag-GFP or pNef-GFP and after four hours, co-cultured with autologous untreated PBMC in absence (RH1-021) or presence (RH1-022, RH1-028 and RH1-029) of 50 IU/ml rIL-2 at PBMC/GFP+ cell ratios of˜150. After overnight incubation, viable GFP+ cells were quantified by flow cytometry and used to calculate percentages of cell-mediated target cell death. Values represent the average+ s.e.m. of triplicates.

FIG. 7: Nucleic acid and amino acid sequences of various HIV and influenza antigens of interest.

### DETAILED DESCRIPTION OF THE INVENTION

### EXAMPLES

### Example 1

### Principle of the Fluorescent Antigen-transfected Target Cytotoxic T-lymphocyte (FATT-CTL) Assay

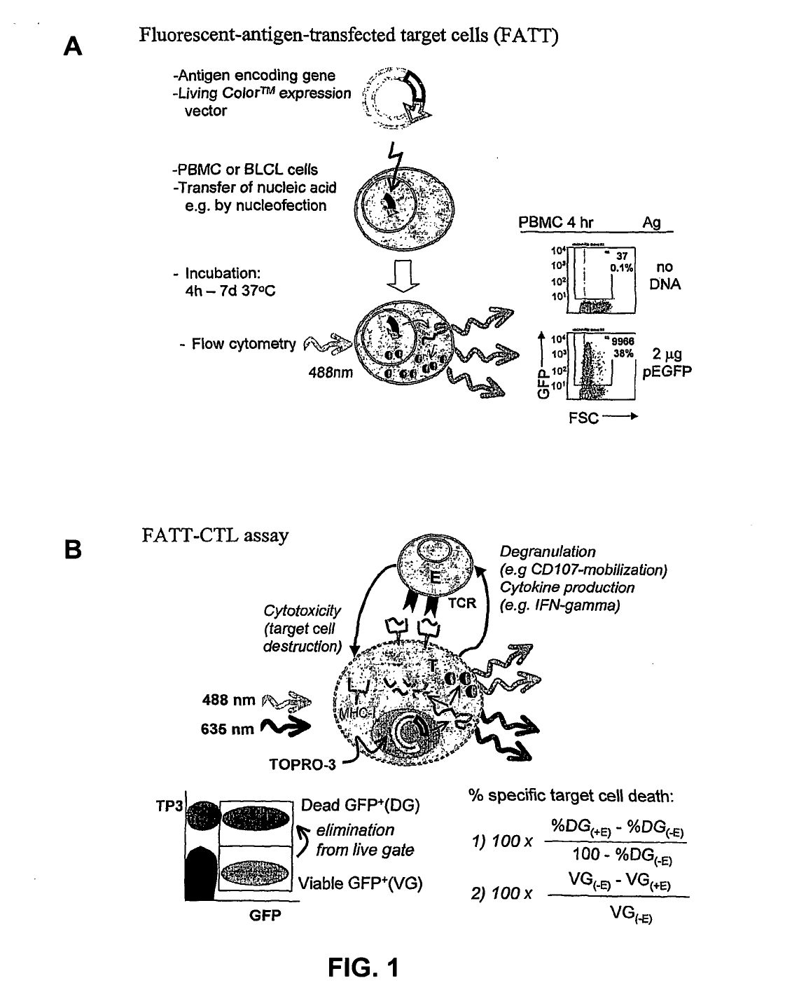

Cytotoxicity is quantified by assessing the elimination of viable cells expressing an antigen of interest associated with a fluorescent reporter molecule. Target cells can be generated by nucleofecting recombinant DNA vectors encoding antigen-fluorescent fusion proteins into PBMC or cell lines (FIG. 1, Panel A). From three to four hours later, expression of the antigen-reporter protein complex can be detected in sufficient cells to set up co-cultures with effector cell populations of interest. Continuous expression of antigen-reporter molecule complexes in the target cells can be detected for several days, depending on the type of target cell and culture conditions.

Elimination of viable antigen-reporter molecule-expressing target cells (T) by cytotoxic effector cells (E) can be detected by any device or method that is designed to detect reporter gene expression, here GFP by flow cytometry (FIG. 1, Panel B). If the total number of target cells does not significantly change during the co-culture period, specific target cell death can be derived from the change in the fraction dead cells (TO-PRO-3+) among the cells expressing the reporter gene, using formula 1 of FIG. 1, Panel B. If incubation periods are long and dying target cells disintegrate, specific target cell elimination can be expressed using formula 2 of FIG. 1, Panel B.

### Example 2

### Cloning of Antigens in Living Colors Vectors (N1)

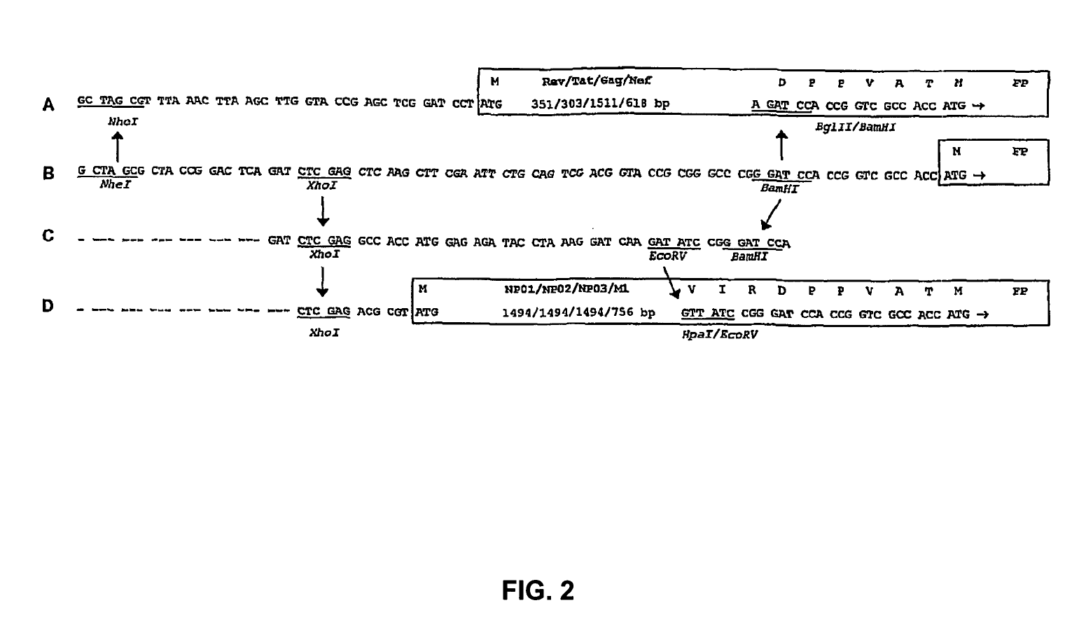

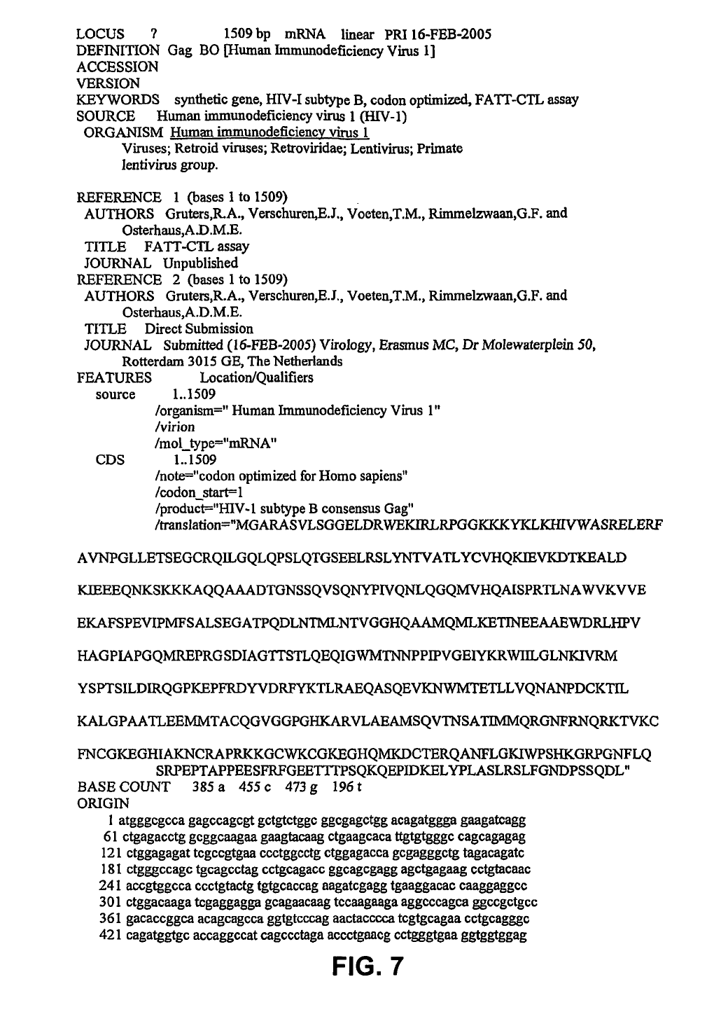

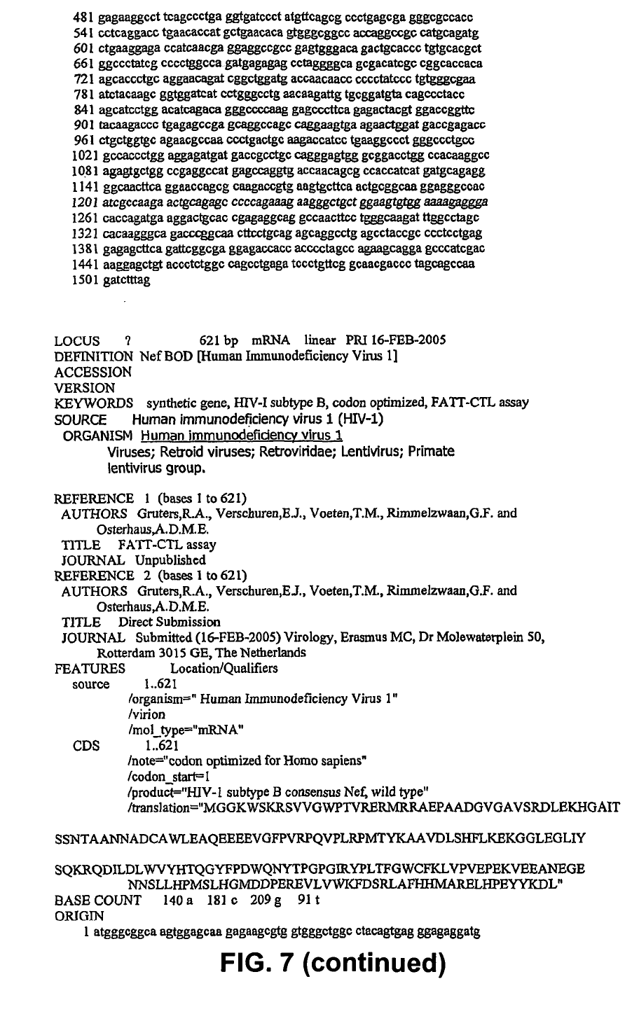

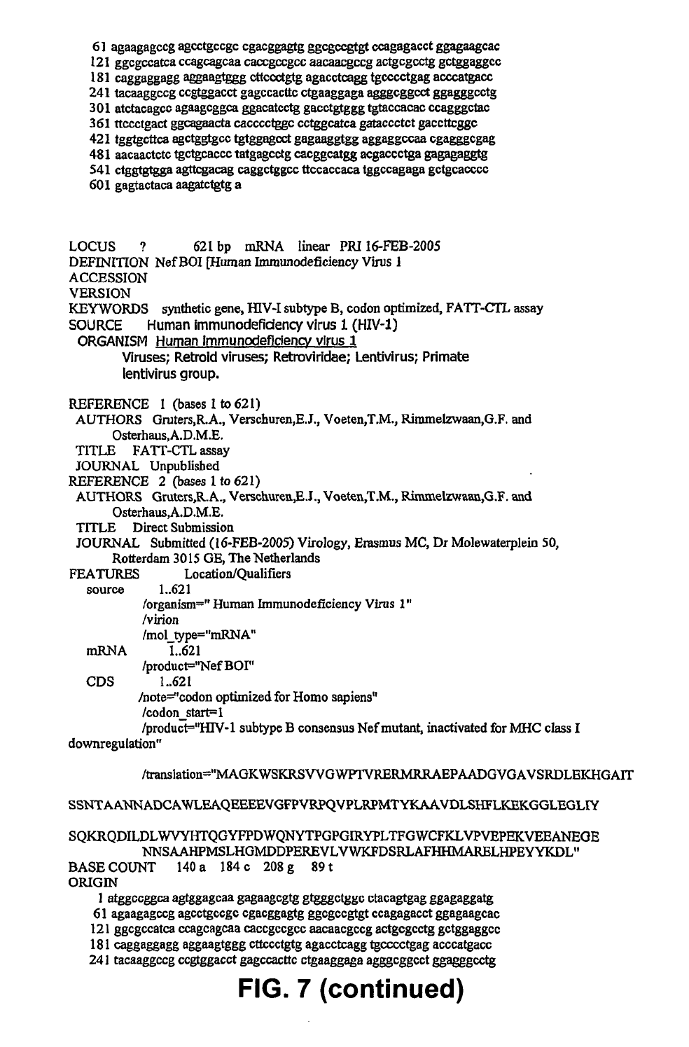

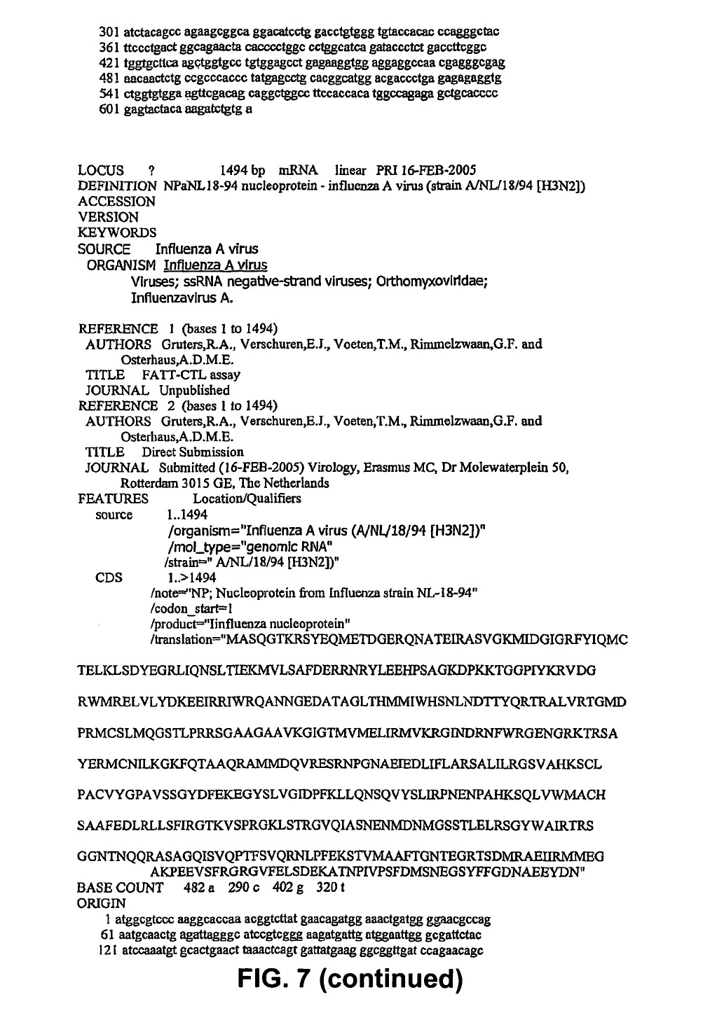

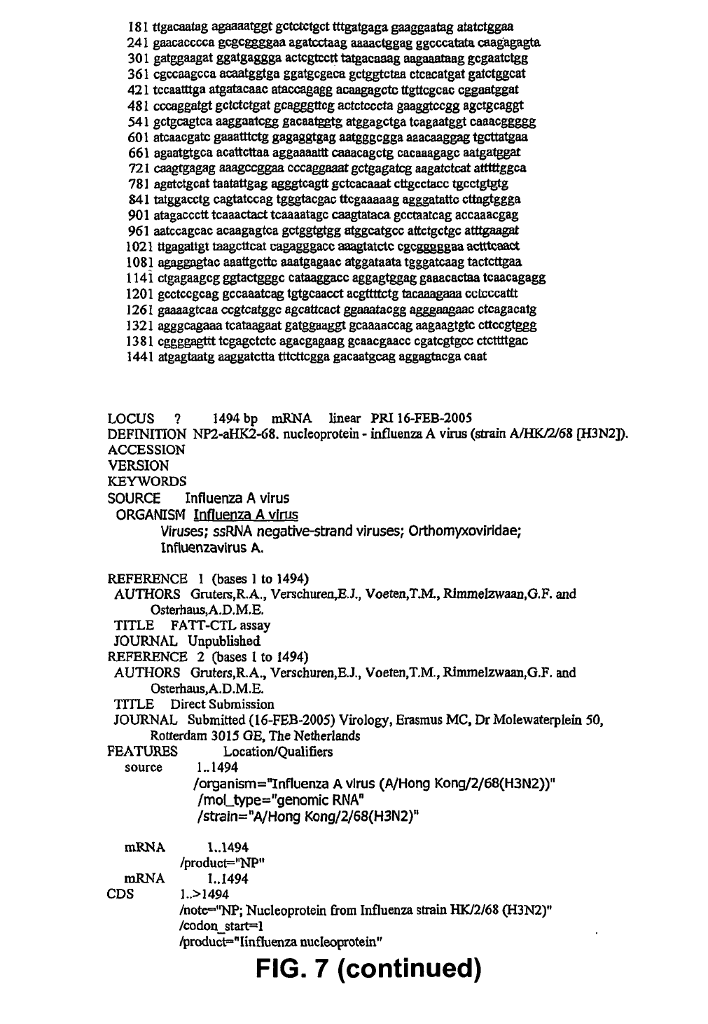

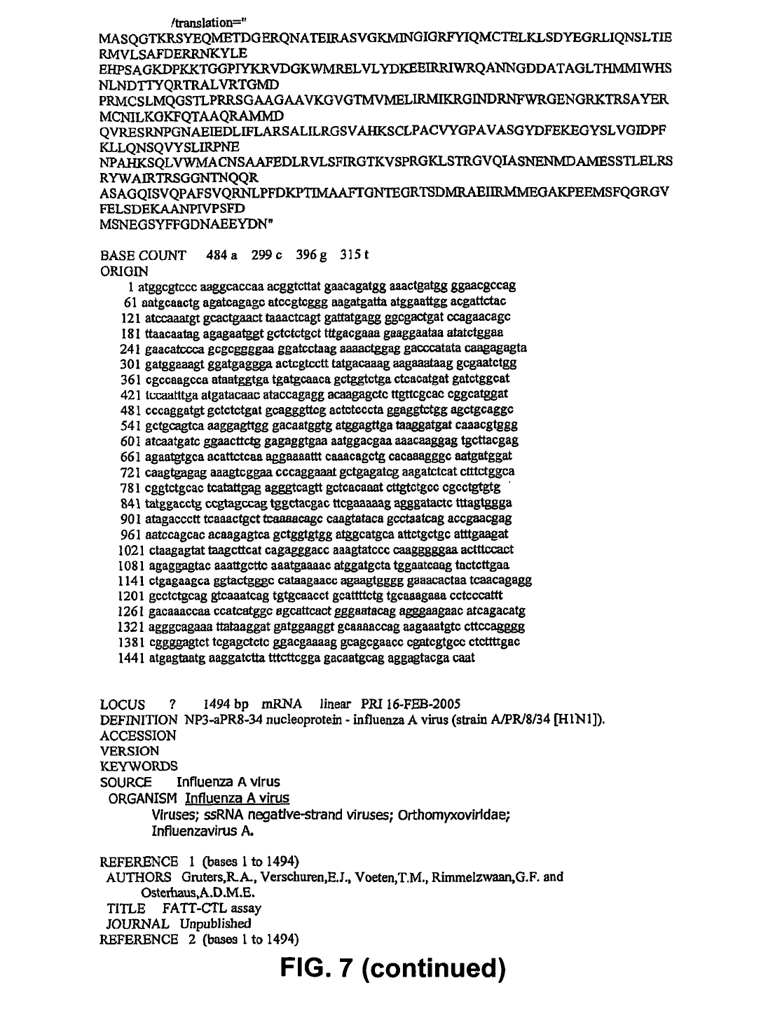

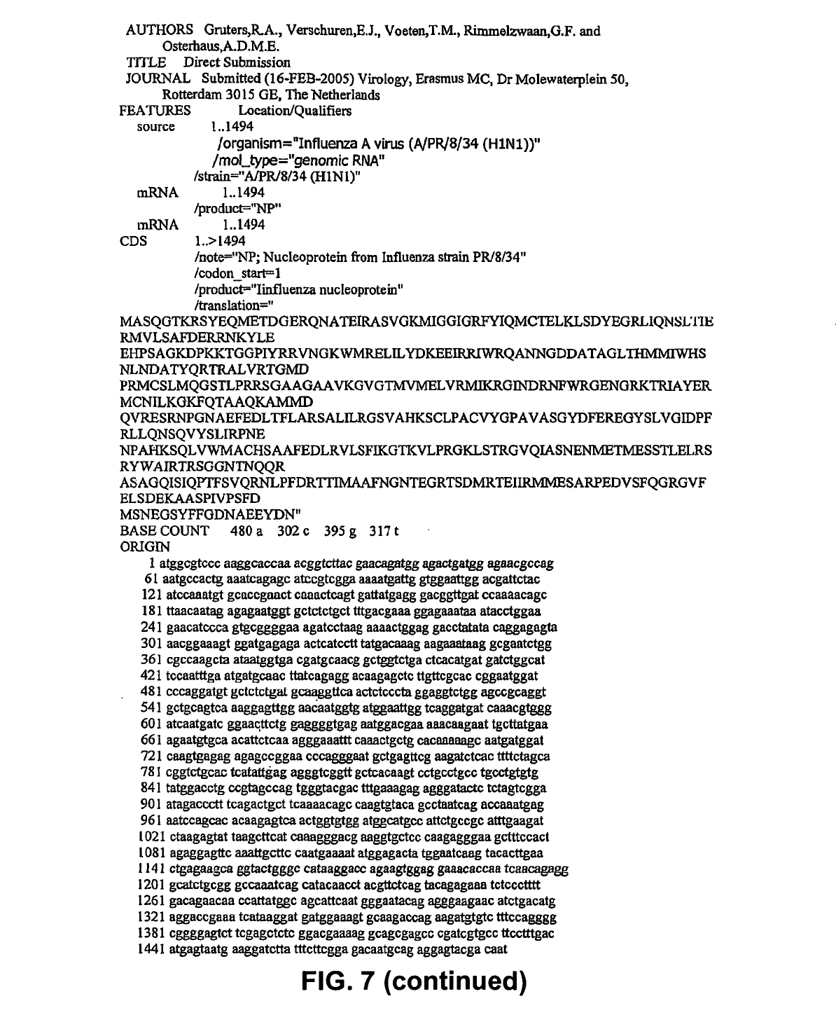

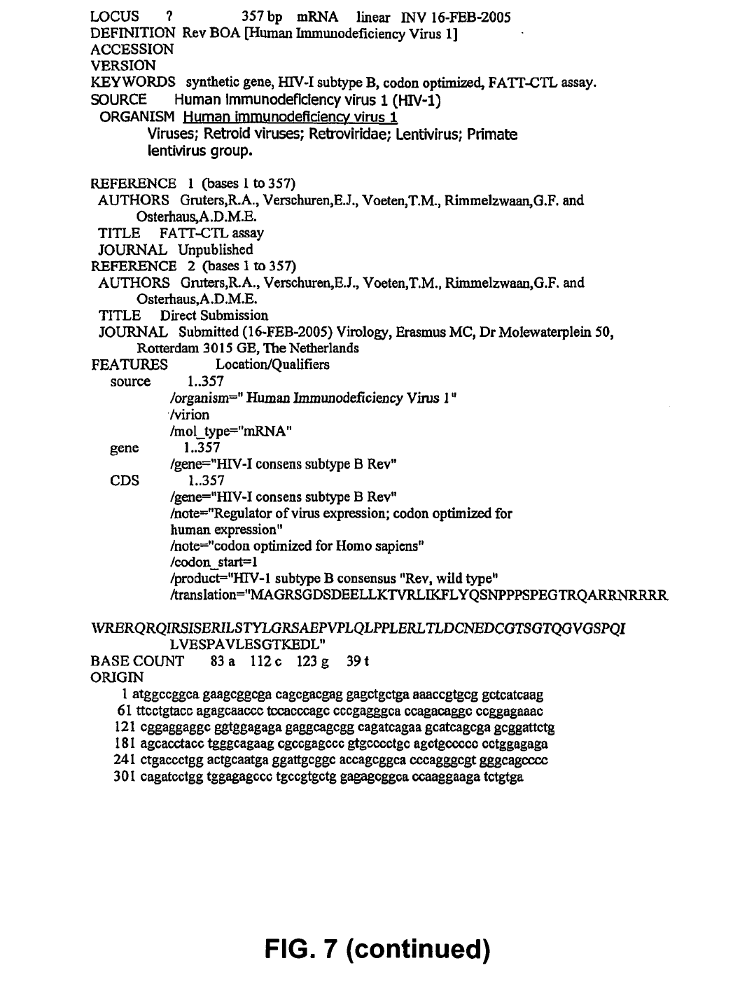

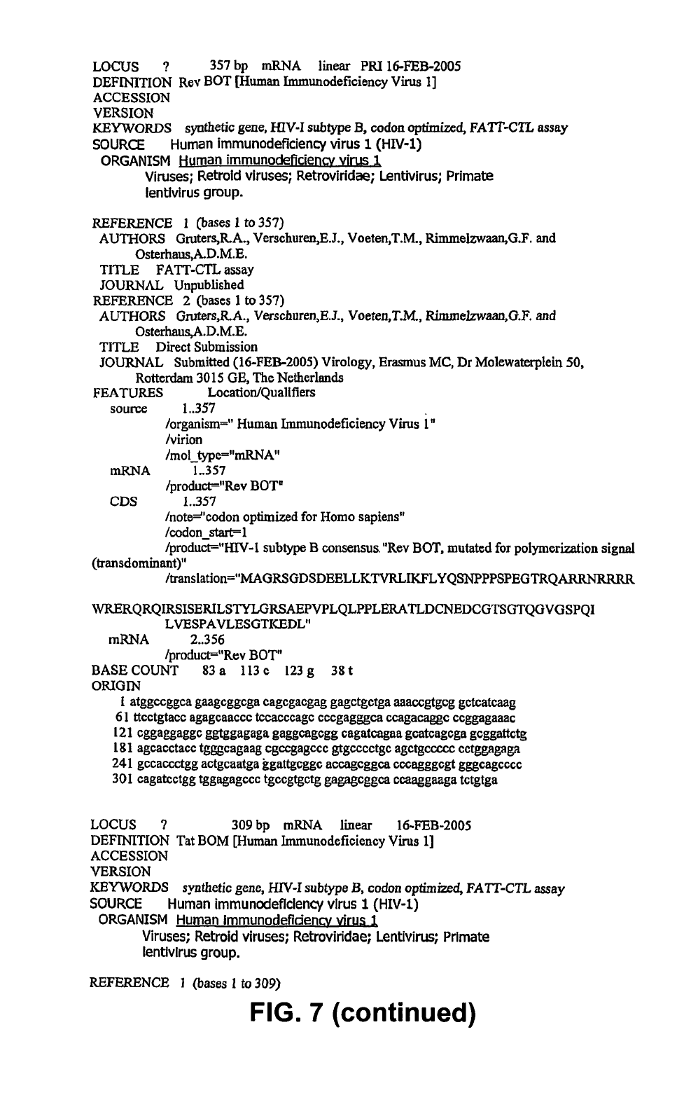

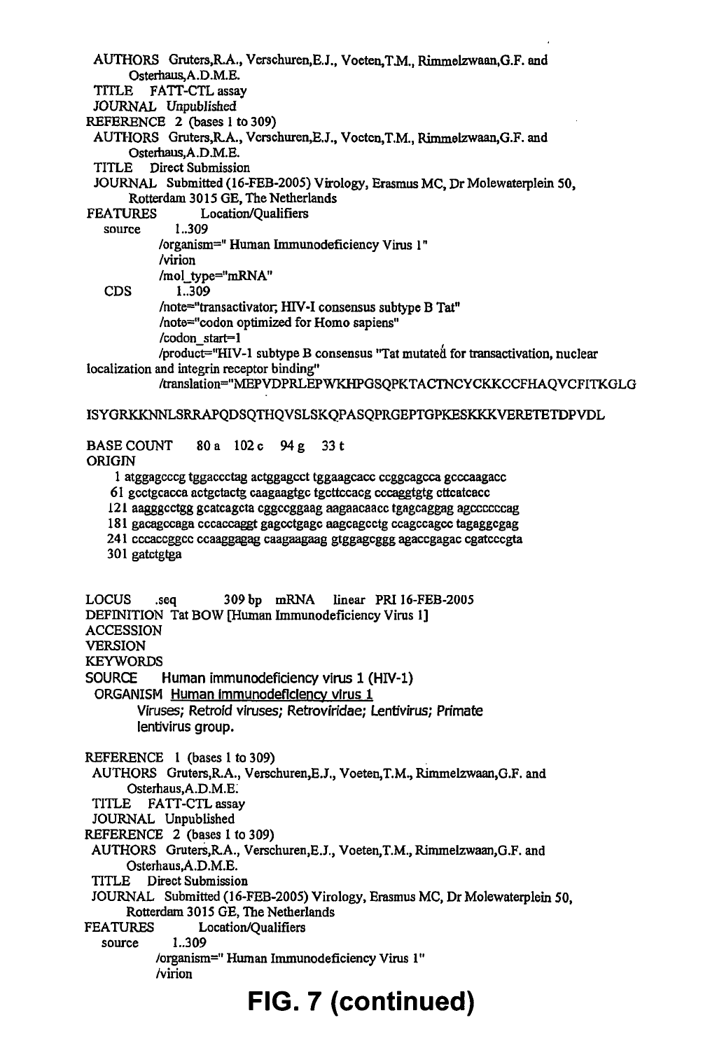

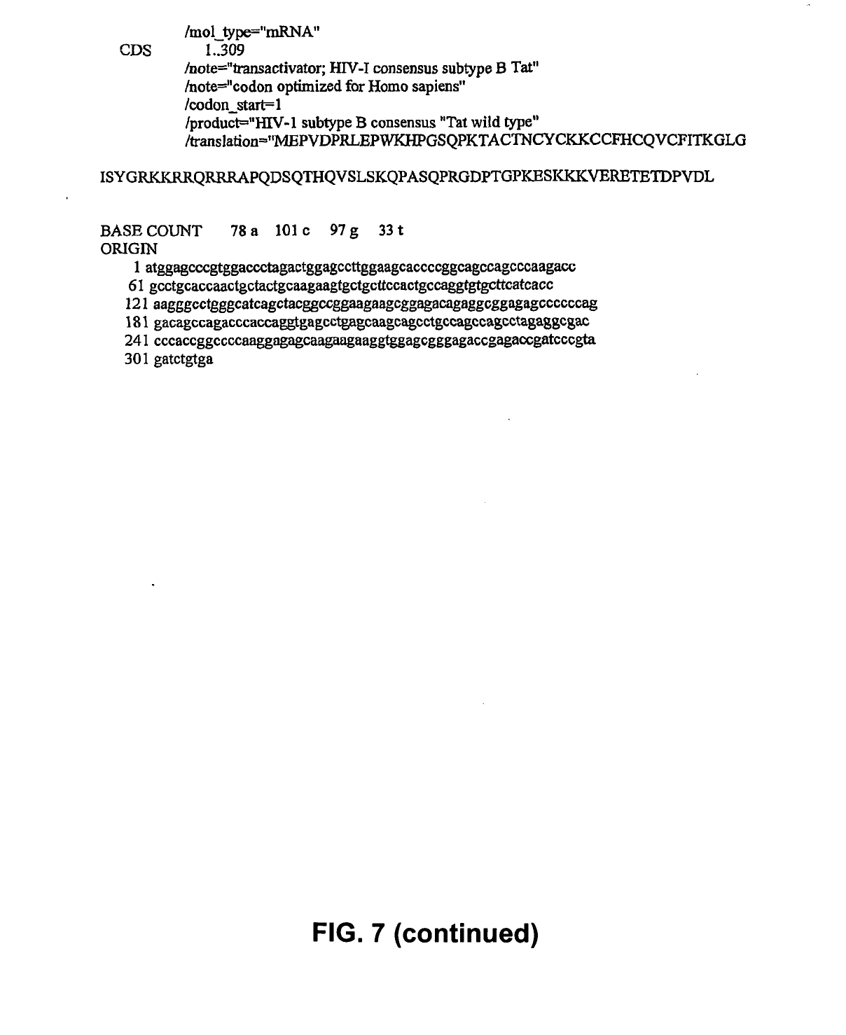

Genes encoding viral proteins of HIV (rev, tat, gag and nef) and influenza A virus nucleoproteins NP01, NP02, NP03 and matrix-proteinM1 were inserted in frame with GFP in the pEGFP-N1 plasmid (BD Biosciences, Erembodegem, Belgium) as depicted in FIG. 2. The sequences of the open reading frames (ORF) are depicted in FIG. 7. Direct cloning of influenza antigens nucleoprotein or matrix protein in pEGFP-N1 was not possible, because the MCS of pEGFP-N1 lacks a restriction site that would result in GFP expressed in frame with NP/Ma. The vector had to be adjusted and simultaneously a GFP construct expressing an Env-epitope was created (pERYL-GFP). For the insert DNA, two primers were designed that code for the Env-epitope ERYLKDQQL followed by an EcoRV restriction site necessary for cloning NP/Ma in frame with GFP. The primers were diluted to 100 pmol/ml and 2 ml of each primer was mixed, heated for five minutes at 95° C. and cooled down to room temperature. Annealing of primers leads to double-strand DNA with sticky ends complementary to the overhanging basepairs after digestion with XhoI and BamHI. After annealing, the sample was diluted to 400 μl with aqua bidest.

After digestion of pEGFP-N1 with XhoI x BamHI, the vector (4.7 kb) was isolated from a 1% agarose gel and used for ligation with the annealed primers as described above. Correct cloning was confirmed by analysis on agarose gel after digestion with EcoRVx NotI (bands 0.7 and 4 kb) and sequencing. Plasmid DNA of pERYL-GFP was digested with XhoI x EcoRV. After DNA precipitation, the vector was dephosphorylated for one hour at 37° C. with alkaline phosphatase (Roche). The phosphatase was inactivated for ten minutes at 72° C. DNA of the pB1-NP and pB1-Ma constructs [1] was digested with XhoI×HpaI. Bands were isolated (NP 1.5 kb, Ma 0.8 kb, pERYL-GFP 4.7 kb) and used for cloning as described above. Correct cloning was confirmed by analysis on agarose gel after digestion with XhoI×NotI (bands 1.5/2.2 kb and 3.9 kb) and sequencing.

### Example 3

### CTL-mediated Killing of Fluorescent Antigen-transfected BLCL Cells and PBMC

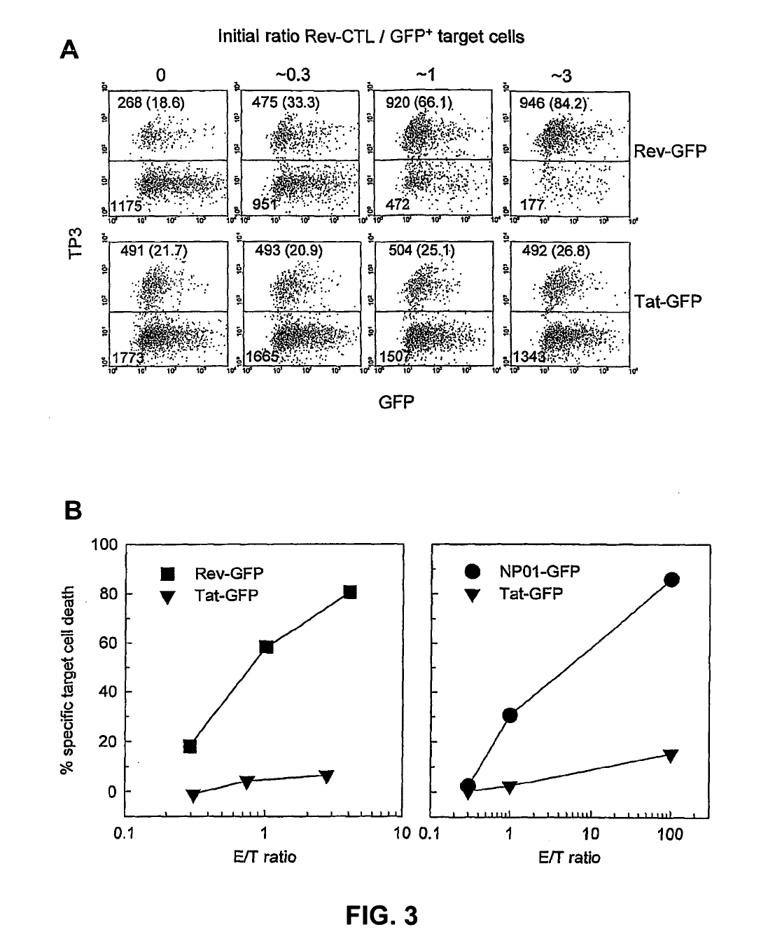

Nucleofection of cells of the EBV-transformed lymphoblastoid cell line (BLCL) B157 with pRev-GFP and pTat-GFP resulted in 50 to 60% GFP+ cells. Antigen processing and presentation of antigen-GFP fusion protein was first assessed by co-culturing pRev-GFP-transfected B157 cells with cells of the Rev-specific CTL clone (709TCC108) at increasing effector-to-target cell (E/T) ratios. pTat-GFP-transfected B157 cells were used as negative control cells. After four hours of incubation, the percentages of dead target cells, i.e., TP3+ GFP+ cells, increased from 20% to 84% in an E/T ratio-dependent fashion. The proportion of non-viable control target cells did not increase (FIG. 3, Section A). After correcting the values for spontaneous background dead cells, the antigen-specific cytolytic activity of the Rev-specific CTL at E/T ratios of ˜0.3, ˜1 and ˜3 were 18%, 58% and 80%, respectively (FIG. 3, Section B, left panel). Next, we explored the use of fresh PBMC as target cells. Nucleofection efficiency of un-stimulated PBMC, or CDS+ depleted PBMC was typically between 30% and 70% (data not shown), which proved to be sufficient for their use as target cells. MHC-class I matched PBMC, nucleofected with pNP01-GFP, were lysed by CTL clone TCC-C10. These data show that BLCL cells as well as PBMC can be used as target cells in the FATT-CTL assay of the invention (FIG. 3, Section B, right panel).

### Example 4

### A Comparison between the Performance of the FATT-CTL Assay and the Classical ⁵¹Cr-release Assay

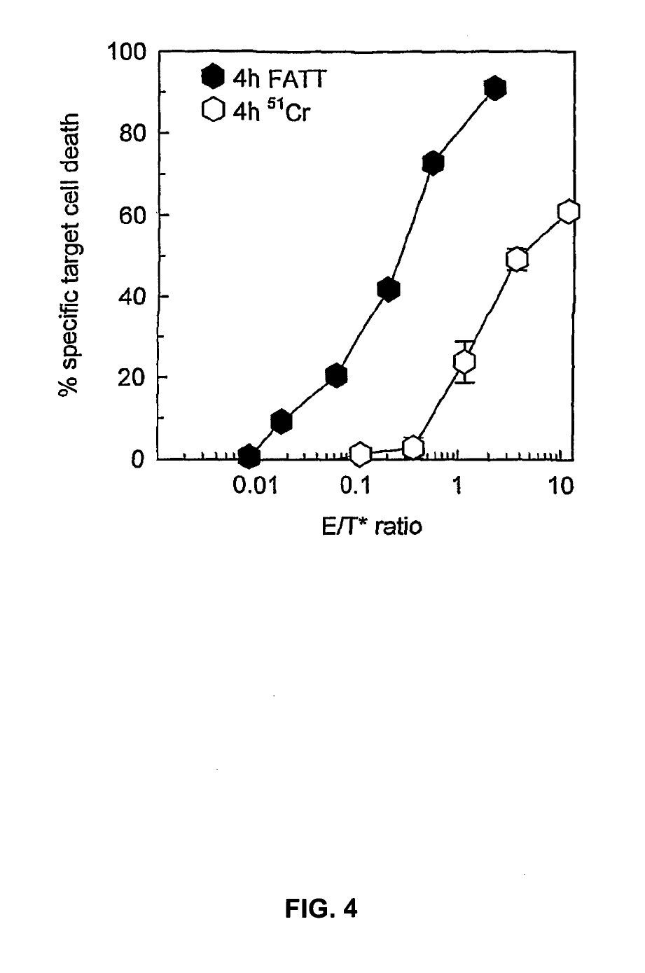

The FATT-CTL assay was compared with a standard ⁵¹Cr-release assay using the same target cell and effector cell populations in both assays. Again, 55 to 60% viable GFP+ events were detected among pRev-GFP- and pTat-GFP-transfected B157 cells. Assuming that CTL epitopes were generated in the GFP+ cells only, this would be the maximum level of specific lysis that could be achieved in the ⁵¹Cr-release assay. Indeed, 58% specific lysis was observed at the highest E/T ratio of 10 (FIG. 4). Using the FATT-CTL assay, more than 90% of the GFP+ cells were lysed by Rev-specific CTL after four hours at the highest E/T ratio. Specific lysis of pTat-GFP+ cells was <3% for all E/T ratios tested in both assays (data not shown). Overall, the FATT-CTL assay was capable of detecting cytotoxicity at significantly lower E/T ratios than the ⁵¹Cr-release assay (FIG. 4). These data show that the FATT-CTL assay detects the cytolytic activity of CTL and that it does so at lower E/T ratios than a classical ⁵¹Cr-release assay.

### Example 5

### CTL Assays with Influenza A Virus-specific CTL and Epitope Variants

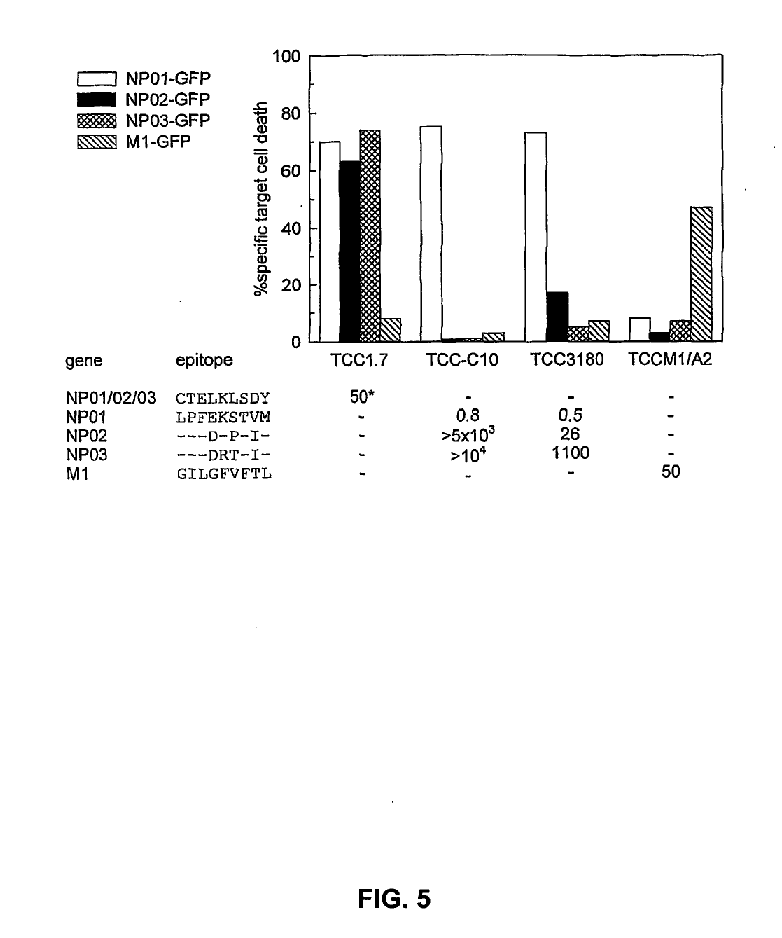

To study the effects of epitope variation on the outcome of FATT-CTL assay, expression vectors encoding various influenza A virus nucleoprotein- and matrix-GFP fusion proteins were constructed. Three vectors were generated using NP-genes derived from distinct influenza virus strains: pNP01-, pNP02-, and pNP03-GFP. These genes contained the same HLA-A*0101 epitope NP44-52 sequence, but differed in the HLA-B*3501 epitope NP418-426 (FIG. 5). pM1-GFP encoded the HLA-A*0201 restricted epitope M158-66. B3180 cells, which express HLA-A*0101, -A*0201 and -B*3501, were nucleofected with the different vectors and co-cultured the following day for three hours with or without cells of three different NP-specific CTL clones, TCC1.7, TCC-C10and TCC3180, or the M1-specific CTL clone M1/A2.

Between 60% and 70% specific lysis was detected among NP01-, NP02- and NP03-GFP+ cells in cultures containing HLA-A*0101-restricted TCC1.7 CTL, specific for the conserved NP44-52 epitope (FIG. 5). The HLA-B*3501-restricted TCC-C10 cells also specifically lysed NP01-GFP+ cells (70%), but not NP02- and NP03-GFP+ cells, in concordance with previously determined EC50 values of the corresponding peptide variants, ˜0.8, >5000 and >10000 nM, respectively [2]. NPO1-GFP+ cells were lysed with similar efficiency by TCC3180 cells that recognized the NP01 peptide variant with an EC50 value of 0.5 nM. The lower avidity of these cells for the NP02-variant peptide, EC50=26nM, was reflected by an approximately four-fold lower level of specific lysis of NP02-GFP+ cells compared to NP01-GFP+ cells (FIG. 5). Cells expressing the NP03-variant, EC50=l lOOnM, were not lysed by TCC3180. The matrix-specific TCC-M1/A2 CTL did not specifically lyse the NP-GFP-expressing cells, but lysed 50% of M1-GFP+ cells (FIG. 5). These data show that the FATT-CTL assay detects CTL-mediated lysis of target cells only if they express the correct epitope sequence, and that the assay detects subtle differences in the functional avidity of the CTL.

### Example 6

### Detecting Antigen-specific Cytotoxicity ex vivo

It was also tested whether the FATT-CTL assay could be applied to detect antigen-specific cell-mediated cytotoxicity directly ex vivo. To this end, PBMC were obtained from four highly active antiretroviral therapy (HAART)-naïve HIV seropositive individuals and four seronegative individuals. Part of the cells was used to generate target cells by nucleofection with pGag-GFP, pNef-GFP, or pEGFP-N1 as a control. Gag and Nef were chosen as antigens because they are among the most frequently recognized. Four hours later, nucleofected and autologous untreated PBMC were co-cultured at PBMC/GFP+ cell ratios of ˜150 with or without rIL-2. After overnight incubation, concentrations of viable GFP+ events were used to calculate antigen-specific target cell elimination.

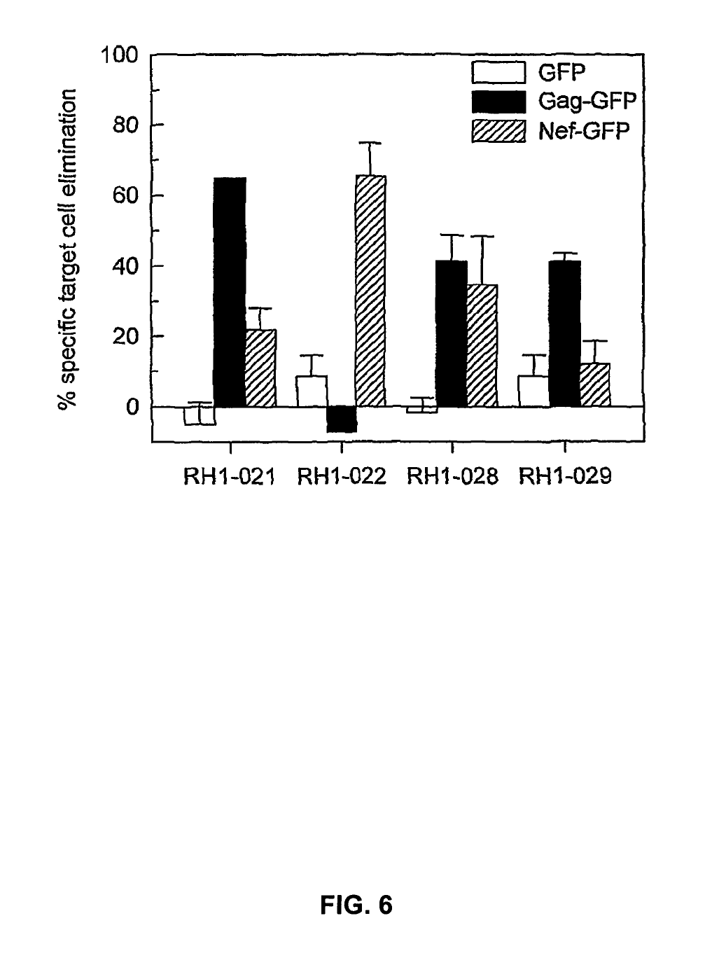

Specific elimination of Gag-GFP- and/or Nef-GFP-, compared to GFP-expressing cells, was observed in the absence (individual RH1-021) or presence (individuals RH1-022, RH1-028 and RH1-029) of exogenous IL-2 (FIG. 6). For individuals RH1-022, RH1-028 and RH1-029, no significant cytotoxicity was observed in the absence of rIL-2 (data not shown). Due to limiting cell numbers, we could not determine cytotoxicity in the presence of IL-2 for individual RH1-021. No Gag- or Nef-specific cytotoxicity, compared to GFP alone, was observed for each of the four seronegative controls, irrespective of the presence of exogenous IL-2 (data not shown). These data illustrate the practical utility of the FATT-CTL assay to directly measure virus-specific CTL activity ex vivo.

Materials and Methods for Studies with Human Materials

Effector Cells

Procedures for the generation and culturing of the CD8+ T-cell clones used have been previously described [2, 4, 5]: 709 TCC108: specific for HIV Rev67-75 epitope SAEPVPLQL; TCC-C10: influenza A, NP418-426 epitope LPFEKSTVM, restricted via HLA-B*3501; TCC3180: influenza A, NP418-426 epitope LPFEKSTVM via HLA-B*3501; TCC1.7: influenza A, NP44-52 epitope CTELKLSDY via HLA-A*0101; TCCM1/A2: influenza A, M58-66 epitope GILGFVFTL via HL-A*0201. The cells were cultured for at least seven days after stimulation with PHA and feeder cells, before use as effector cells in CTL assays.

Vectors

The cloning strategy for the construction of vectors pRev-GFP, pTat-GFP, pGag-GFP, pNef-GFP, pNPO1-GFP, pNP02-GFP, pNP03-GFP and pM1-GFP is depicted in FIG. 2. Genes were cloned into the multiple cloning site of Living Colors™ vectors pEGFP-N1, pDsRedExpress-N1 and pHcRedl-N1/1, in frame with the fluorescent protein (FP) ORF using the indicated restriction enzymes. By omitting the stop-codon of the cloned genes, read-through of the fluorescent gene was achieved. HIV genes were codon-optimized consensus subtype B synthetic genes (GeneArt, Regensburg, Germany). Influenza genes were derived from: NP strain A/NL/18/94 (NP01), NP strain A/HK/2/68 (NP02), NP strain A/PR/8/34 (NP03), M1 strain A/NL/18/94 (M1) [6]. Inserts were sequenced to confirm that no errors had been introduced and that they were expressed in frame with the fluorescent protein ORF. Sequences (see FIG. 7) have been submitted to Genebank.

Target cells

Two EBV-transformed B-lymphoblastoid cell lines (BLCL), B157 and B3180, were used as source of autologous or HLA-matched target cells for the CTL clones. Antigen expression was achieved by transfecting BLCL cells with plasmid DNA vectors using the Amaxa Nucleofector™ technology (Amaxa, Cologne, Germany) according to the manufacturers' instructions. Briefly, one to 2×10⁶ cells in logarithmic growth phase were resuspended in 100 μl nucleofection buffer containing 2 to 4 μg DNA, and subjected to one of the electroporation programs. Subsequently, cells were cultured overnight in a final volume of 2 to 4 ml RPMI1640 supplemented with antibiotics and 10% Fetal Calf Serum (RIOF) at 37° C. 5% CO₂. All buffers and programs of the Cell Line Optimization Nucleofector™ kit (Amaxa) were tested, and the combination of buffer V with program P-16 resulted in the highest concentration of viable GFP-expressing cells, combined with high overall viability, i.e., 50% after 24 hours (data not shown). Target cells for the ex vivo FATT-CTL assay were generated by nucleofecting freshly isolated PBMC using the optimized Human T-Cell Nucleofector™ kit (Amaxa), as described below.

FATT-CTL assay (four hours)

Target cells were washed and co-cultured with effector cells at increasing effector-to-target cell (E/T) ratios in 200 μl R10F, at 37° C. 5% CO₂ for three to four hours. Cells were transferred to wells or tubes containing 5 μl EDTA (3 mM final concentration) to reduce the number of cell to cell conjugates, and 5 μl TO-PRO-3 iodide (TP3; 25 nM final concentration, Molecular Probes, Leiden, The Netherlands) to discriminate viable and non-viable cells [7]. In some experiments, EDTA/TP3-treated cells were cooled on ice and stained with anti-CD8-PE (BD Biosciences, Erembodegem-Aalst, Belgium) for 20 minutes prior to acquisition. The ^{5l}Cr-release assay was performed as described previously [4]. Samples were acquired on a FACS-Calibur (BD Biosciences) for a fixed period of 60 seconds per sample. The forward scatter (FSC) acquisition threshold was set to include non-viable events. Debris was excluded by gating in FSC-TP3 dotplots during data analyses. The flow rate was plotted in a Time-Event histogram and generally proved to be constant in each of the samples per experiment. If not, we defined a region to select a shared period of constant flow rate. A region to exclude GFP events was defined in GFP-TP3 or GFP-FL3 dotplots of the data acquired from cultures containing BLCL cells that had not been nucleofected. GFP+ events derived from cultures containing nucleofected BLCL cells were displayed in FSC-TP3 or GFP-TP3 dotplots to define viable GFP+ (VG) events, i.e., TP3-, and non-viable or dead GFP+ (DG) events, i.e., TP3+ (see FIG. 2, Line A). Percentages of dead GFP+ events (%DG) were calculated by the formula: 100 * (number of DG)/(number of VG+number of DG). CTL-mediated target cell death was calculated with the formula 100 * (% DG_{+E}−% DG_{-E})/(100−% DG_{-E}) where+ E and −E denotes the presence or absence of effector cells in the cultures, respectively.

Ex vivo FATT-CTL Assay (18 to 24 hours)

PBMC were isolated by density centrifugation (Lymphoprep™, Nycomed, Oslo, Norway) of heparin blood (28 to 30 ml) obtained from four HIV-1 seropositive individuals visiting the ErasmusMC in Rotterdam, The Netherlands, who received no antiviral treatment, had CD4 counts of more than 300 cells/mm³ and a viral load between 50 and 1×10⁵ RNA copies/ml. As controls, we isolated PBMC from buffy coats obtained from healthy blood donors. Freshly isolated PBMC (2×10⁶ cells/cuvette) were nucleofected with plasmid DNA vectors (2 μg) using the Human T-Cell Nucleofector™ kit (Amaxa) according to the manufacturer's instructions, and incubated in 1.5 to 2.0 ml R10F medium at 37° C., 5% CO₂ in a humidified incubator. Four hours later, we determined the concentration of viable GFP+ events in a 50 μl sample using TruCOUNT tubes (BD Biosciences) and initiated co-cultures of ˜3000 GFP+ events per well with untreated PBMC at a PBMC/GFP+ cell ratio of 150 (triplicates) in 96 micro-well Thermo-Fast 96 detection plates (ABgene, Surrey, UK) in a total volume of 200 μl per well with or without rIL-2 (50 IU/ml). After overnight incubation, the cultures were transferred to micronic tubes containing 5 μl EDTA (3 mM final concentration) and 5 μl TP3 (25 nM final concentration), incubated for 20 minutes at 37° C., transferred to melting ice and acquired on a FACS-Calibur within two hours. To prevent event count rates exceeding 2000 total event/sec, we set an FL1-threshold during acquisition to exclude the majority of GFP events, in addition to an FSC-threshold to exclude debris. Because many killed GFP+ cells can no longer be detected as TP3+GFP+ events after an overnight incubation period (data not shown), we used the difference between the number of viable GFP+ (VG) events in cultures with (VG_{+E}) and without (VG_{-E}) effector PBMC to calculate the percentage of PBMC-mediated antigen-specific target cell death, i.e., 100* (VG_{-E}−VG_{+E})/VG_{-E}.

### REFERENCES

1. Voeten J. T., G. F. Rimmelzwaan, N. J. Nieuwkoop, K. Lovgren-Bengtsson, and A. D. Osterhaus. Introduction of the haemagglutinin tran.smembrane region in the influenza virus matrix protein facilitates its incorporation into ISCOM and activation of specific CD8(+) cytotoxic T-lymphocytes. Vaccine 2000; 19(4-5):514-22.
2. Boon A. C., G. de Mutsert, D. van Baarle, D. J. Smith, A. S. Lapedes, R.A. Fouchier, et al. Recognition of homo- and heterosubtypic variants of influenza A viruses by human CD8+ T-lymphocytes. J. Immunol. 2004; 172(4):2453-60.
3. Siebelink K. H., I. H. Chu, G. F. Rimmelzwaan, K. Weijer, A. D. Osterhaus, and M. L. Bosch. Isolation and partial characterization of infectious molecular clones of feline immunodeficiency virus obtained directly from bone marrow DNA of a naturally infected cat. J. Virol. 1992; 66(2):1091-7.
4. Van Baalen C. A., M. Schutten, R. C. Huisman, P. H. Boers, R. A. Gruters, and A. D. Osterhaus. Kinetics of antiviral activity by human immunodeficiency virus type 1-specific cytotoxic T-lymphocytes (CTL) and rapid selection of CTL escape virus in vitro. J. Virol. 1998; 72(8):6851-7.
5. Boon A. C., G. de Mutsert, R. A. Fouchier, K. Sintnicolaas, A. D. Osterhaus, and G. F. Rimmelzwaan. Preferential HLA usage in the influenza virus-specific CTL response. J. Immunol. 2004; 172(7):4435-43.
6. Voeten J. T., G. F. Rimmelzwaan, N. J. Nieuwkoop, R. A. Fouchier, and A. D. Osterhaus. Antigen processing for MHC class I restricted presentation of exogenous influenza A virus nucleoprotein by B-lymphoblastoid cells. Clin. Exp. Immunol. 2001; 125(3):423-31.
7. Lee-MacAry A. E., E. L. Ross, D. Davies, R. Laylor, J. Honeychurch, M. J. Glennie, et al.

Development of a novel flow cytometric cell-mediated cytotoxicity assay using the fluorophores PKH-26 and TO-PRO-3 iodide. J. Immunol. Methods 2001; 252(1-2):83-92.
8. Koksoy S., A. J. Phipps, K. A. Hayes, and L. E. Mathes. SV40 Immortalization of feline fibroblasts as targets for MHC-restricted cytotoxic T-cell assays. Vet. Immunol. Immunopathol. 2001; 79(3-4):285-95.

1. A method for detecting cytolytic activity of either cells or a substance against a population of target cells, said method comprising:
providing target cells with a first nucleic acid sequence encoding a reporter molecule and a second nucleic acid sequence encoding an antigen of interest;
co-culturing said target cells with a sample containing cells or a substance suspected of having cytolytic activity; and
detecting the viability of target cells provided with the reporter molecule.

2. The method according to claim 1, wherein said reporter molecule is selected from the group consisting of a fluorescent polypeptide, GFP, YFP, CFP, EGFP, EYFP, ECFP, HcRed, DsRed, and a cell surface marker.

3. The method according to claim 1, wherein said antigen of interest is selected from the group consisting of a viral antigen, a bacterial antigen, a parasitic antigen, and a tumor antigen.

4. The method according to claim 1, wherein said first and second nucleic acid sequences are cloned, in frame, to encode a fusion protein of said antigen of interest with said reporter molecule.

5. The method according to any claim 1, wherein said target cells are primary cells or cells from a cell line.

6. The method according to claim 1, wherein said target cells are provided with the first and second nucleic acid sequences using a method selected from the group consisting of cell electroporation, cell transfection, nucleofection, and infection with a recombinant pathogen of interest expressing a reporter molecule.

7. The method according to claim 1, wherein the viability of target cells is detected using fluorescence detection equipment, preferably using fluorescence activated cell sorting (FACS), Immune Fluorescence (IF) analysis or a Fluorometer.

8. The method according to claim 7, wherein the viability of target cells is detected using fluorescence activated cell sorting (FACS), Immune Fluorescence (IF) analysis or a Fluorometer.

9. The method according to claim 1, further comprising:
detecting a cell surface marker that is specific for target cells or specific for a cytotoxic T lymphocyte (CTL) to distinguish between target cells and CTL.

10. The method according to claim 9 wherein said cell surface marker is CD8.

11. The method according to claim 1, further comprising:
detecting the ability of target cells to take up a viability dye, preferably a viability dye which stains nucleic acid, more preferably TO-PRO-3 iodide.

12. A method of testing a mammal to determine if the mammal has acquired or retains immunity from a previous vaccination, immunization and/or disease exposure, said method comprising:
taking a biological sample from the mammal, and
analyzing an analyte comprising the biological sample with the method according to claim 1 so as to detect cytolytic activity.

13. A kit of parts for detecting cytolytic activity of either cells or a substance against a population of target cells, said kit of parts comprising:
an expression vector with a first nucleic acid sequence encoding a reporter molecule that can stably associate with a target cell's plasma membrane,
a multiple cloning site allowing for subcloning of a second nucleic acid sequence encoding an antigen of interest and regulatory elements needed to express the sequences in a target cell, and
means for transfecting a target cell with said vector.

14. A kit of parts for detecting cytolytic activity of either cells or a substance against a population of target cells, said kit of parts comprising:
a first expression vector comprising a first nucleic acid sequence encoding a reporter molecule that can stably associate with a target cell's plasma membrane,
a second expression vector comprising a second nucleic acid sequence encoding an antigen of interest,
wherein said first and second expression vectors comprise regulatory elements needed to express the sequences in a target cell, and
means for transfecting a target cell with said first and second expression vectors.

15. The kit of parts of claim 13, wherein said antigen of interest is selected from the group consisting of a viral antigen, a bacterial antigen, a parasitic antigen, a tumor antigen, an HIV-1 antigen, and an influenza antigen.

16. The kit of parts of claim 14, wherein said antigen of interest is selected from the group consisting of a viral antigen, a bacterial antigen, a parasitic antigen, a tumor antigen, an HIV-1 antigen, and an influenza antigen.

17. The kit of parts of claim 13, further comprising:
at least one detectable reagent capable of recognizing a cell surface marker that is specific for target cells or specific for cytolytic cells,
a viability dye, or
both at least one detectable reagent capable of recognizing a cell surface marker that is specific for target cells or specific for cytolytic cells and a viability dye.

18. The kit of parts of claim 14, further comprising:
at least one detectable reagent capable of recognizing a cell surface marker that is specific for target cells or specific for cytolytic cells,
a viability dye, or
both at least one detectable reagent capable of recognizing a cell surface marker that is specific for target cells or specific for cytolytic cells and a viability dye.

19. The kit of parts of claim 15, further comprising:
at least one detectable reagent capable of recognizing a cell surface marker that is specific for target cells or specific for cytolytic cells,
a viability dye, or
both at least one detectable reagent capable of recognizing a cell surface marker that is specific for target cells or specific for cytolytic cells and a viability dye.

20. The kit of parts of claim 16, further comprising:
at least one detectable reagent capable of recognizing a cell surface marker that is specific for target cells or specific for cytolytic cells,
a viability dye, or
both at least one detectable reagent capable of recognizing a cell surface marker that is specific for target cells or specific for cytolytic cells and a viability dye.
